SEQUENCE LISTING (7 sequences)

SEQ ID NO: 1 - protein, 29 aa, Artificial Sequence
  Description of Artificial Sequence Synthetic peptide
MMSFVSLLLVGILFYATEAEQLTKCEVFQ

SEQ ID NO: 2 - protein, 4 aa, Artificial Sequence
  Description of Artificial Sequence Synthetic peptide
KEDL

SEQ ID NO: 3 - protein, 8 aa, Artificial Sequence
  Description of Artificial Sequence Synthetic peptide
PPKKKRKV

SEQ ID NO: 4 - protein, 25 aa, Artificial Sequence
  Description of Artificial Sequence Synthetic peptide
MLSLRQSIRFFKPATRTLCSSRYLL

SEQ ID NO: 5 - protein, 6 aa, Artificial Sequence
  Description of Artificial Sequence Synthetic 6xHis tag
HHHHHH

SEQ ID NO: 6 - protein, 12 aa, Artificial Sequence
  Description of Artificial Sequence Synthetic peptide
QEGFSDLWKLLS

SEQ ID NO: 7 - protein, 12 aa, Artificial Sequence
  Description of Artificial Sequence Synthetic peptide
QEGFSDLWKLLS

(19) United States
(12) Patent Application Publication
(10) Pub. No.: US 2013/0123196 A1
(43) Pub. Date: May 16, 2013

(54) THIOETHER-, ETHER-, AND ALKYLAMINE-LINKED HYDROGEN BOND SURROGATE PEPTIDOMIMETICS

(71) Applicants: Paramjit S. Arora, Huntington, NY (US); Andrew Mahon, New York, NY (US)

(72) Inventors: Paramjit S. Arora, Huntington, NY (US); Andrew Mahon, New York, NY (US)

(73) Assignee: NEW YORK UNIVERSITY, NEW YORK, NY (US)

(21) Appl. No.: 13/601,648

(22) Filed: Aug. 31, 2012

Related U.S. Application Data
(60) Provisional application No. 61/529,414, filed on Aug. 31, 2011.

(51) Int. Cl. C07K 7/54

(52) U.S. Cl. CPC C07K 7/54
USPC 514/21.1; 530/323; 435/375

(57) ABSTRACT

Provided herein are peptidomimetics and their salts having a stable, internally constrained protein secondary structure containing a thioether-, ether-, or alkylamine-linked hydrogen bond surrogate; compositions containing at least one of these, and methods of making and using these.

### CROSS REFERENCE TO RELATED APPLICATION

This application claims priority to U.S. Provisional Application No. 61/529,414, filed on Aug. 31, 2011, that is incorporated herein by reference in its entirety.

### STATEMENT REGARDING FEDERALLY SPONSORED RESEARCH

This invention was made with government support under grant number R01GM073943 awarded by the National Institutes of Health. The government has certain rights in this invention.

### FIELD OF THE INVENTION

Inventive embodiments herein are directed generally, but not limited to, the design of and/or to protein-targeting properties of thioether-, ether-, and alkylamine-linked hydrogen bond surrogate peptidomimetics and their salts, to these peptidomimetics and their salts, to compositions containing at least one of these, to methods of making these, and to methods of using these.

### BACKGROUND OF THE INVENTION

Protein secondary structures include β-sheets/β-hairpins, π-helices, 3₁₀-helices, and α-helices.

The α-helix is the most common element of protein secondary structure and participates widely in fundamental biological processes, including highly specific protein-protein and protein-nucleic acid interactions. Molecules that can predictably and specifically disrupt these interactions would be invaluable as tools in molecular biology, and, potentially, as leads in drug development (Kelso et al., J. Am. Chem. Soc. 126:4828-4842 (2004); Schafmeister et al., J. Am. Chem. Soc., 122:5891-5892 (2000); Austin et al., J. Am. Chem. Soc. 119:6461-6472 (1997); Phelan et al., J. Am. Chem. Soc. 119:455-460 (1997); Osapay et al., J. Am. Chem. Soc. 114:6966-6973 (1992); Kemp et al., J. Org. Chem. 56:6672-6682(1991); Jackson et al., J. Am. Chem. Soc. 113:9391-9392 (1991); Ghadiri et al., J. Am. Chem. Soc. 112:1630-1632 (1990); Felix et al., Int. J. Pept. Protein Res. 32:441-454 (1988)). Exposed α-helices on the surfaces of proteins are also often involved in recognition of other biomolecules. Peptides composed of less than fifteen residues corresponding to these α-helical regions typically do not remain helical once excised from the protein environment. Short peptides (<15 residues) that can adopt α-helical structure are expected to be useful models, for example, for the design of bioactive molecules and for studying aspects of protein folding.

Several strategies have been reported for the preparation of stabilized α-helices (Andrews et al., “Forming Stable Helical Peptides Using Natural and Artificial Amino Acids,” Tetrahedron 55:11711-11743 (1999)). These methods include incorporation of nonnatural amino acids (Lyu et al., “Alpha-helix Stabilization by Natural and Unnatural Amino Acids with Alkyl Side Chains,” Proc. Nat'l Acad. Sci. 88:5317-5320 (1991); Kaul et al., “Stereochemical Control of Peptide Folding,” Bioorg. Med. Chem. 7:105-117 (1999)), capping motifs (Austin et al., “Template for Stabilization of a Peptide Alpha-helix: Synthesis and Evaluation of Conformational Effects by Circular Dichroism and NMR,” J. Am. Chem. Soc. 119:6461-6472 (1997); Lyu et al., “Capping Interactions in Isolated Alpha Helices: Position-dependent Substitution Effects and Structure of a Serine-capped Peptide Helix,” Biochemistry 32:421-425 (1993); Chakrabartty et al., “Helix Capping Propensities in Peptides Parallel Those in Proteins,” Proc. Nat'l Acad. Sci. U.S.A. 90:11332-11336 (1993); Kemp et al., “Studies of N-Terminal Templates for Alpha-helix Formation—Synthesis and Conformational-analysis of (2s,5s,8s,11s)-1-acetyl-1,4-diaza-3-keto-5-carboxy-10-thiatricyclo[2.8.1.0(4,8)]tridecane (Ac-Hell-Oh),” J. Org. Chem. 56:6683-6697 (1991)), salt-bridges (Bierzynski et al., “A Salt Bridge Stabilizes the Helix Formed by Isolated C-Peptide of RNase A,” Proc. Nat'l Acad. Sci. U.S.A. 79:2470-2474 (1982)), metal ion chelation (Kelso et al., J. Am. Chem. Soc., 126:4828-4842 (2004); Kelso et al., “A Cyclic Metallopeptide Induces Alpha Helicity in Short Peptide Fragments of Thermolysin,” Angew. Chem. Int. Ed. Engl. 42:421-424 (2003); Ruan et al., “Metal-ion Enhanced Helicity in Synthetic Peptides Containing Unnatural, Metal-ligating Residues,” J. Am. Chem. Soc. 112:9403-9404 (1990); Ghadiri, J. Am. Chem. Soc., 112:1630-1632 (1990)), and covalent side chain linkers such as disulfide (Jackson et al., “A General Approach to the Synthesis of Short Alpha-helical Peptides,” J. Am. Chem. Soc. 113:9391-9392 (1991)), lactam (Phelan et al., “A General Method for Constraining Short Peptides to an Alpha-helical Conformation,” J. Am. Chem. Soc. 119:455-460 (1997); Bracken et al., J. Am. Chem. Soc. 116:6431-6432 (1994); Osapay et al., J. Am. Chem. Soc., 114:6966-6973 (1992); Felix et al., Int. J. Pept. Protein Res. 32:441-454 (1988)), and hydrocarbon bridges (Schafmeister et al., “An All-hydrocarbon Cross-linking System for Enhancing the Helicity and Metabolic Stability of Peptides,” J. Am. Chem. Soc. 122:5891-5892 (2000); Blackwell et al., “Highly Efficient Synthesis of Covalently Cross-linked Peptide Helices by Ring-closing Metathesis,” Angew. Chem. Int. Ed. Engl. 37:3281-3284 (1998)). Stabilization of the α-helix structure with these strategies is typically context dependent (Geistlinger et al., “An Inhibitor of the Interaction of Thyroid Hormone Receptor Beta and Glucocorticoid Interacting Protein,” J. Am. Chem. Soc. 123:1525-1526 (2001); McNamara et al., “Peptides Constrained by an Aliphatic Linkage between Two C(alpha) Sites: Design, Synthesis, and Unexpected Conformational Properties of an i,(i+4)-Linked Peptide,” Org. Chem. 66:4585-4594 (2001)). More importantly, however, these strategies typically block solvent-exposed surfaces of the target α-helices, or restrict or replace important side chain functionalities from the putative α-helices.

Thus, there remains a need for identifying a general method for the synthesis of highly stable internally-constrained peptide structures, such as short α-helical peptides, with strict preservation of the helix surfaces. Stabilized α-helices and helix mimetics have emerged as powerful antagonists of model protein-protein interactions (Edwards & Wilson, Amino Acids 1-12 (2011); Patgiri et al., Nature Chem. Biol. 7:585-87 (2011); Henchey et al., J. Am. Chem. Soc. 132:941-43 (2010); Moellering et al., Nature 462:182-88 (2009); Walensky et al., Science 305:1466-70 (2004); Harrison et al., Proc. Nat'l Acad. Sci. USA 107:11686-91 (2010); Home et al., Proc. Nat'l Acad. Sci. USA 106:14751-56 (2009); Home & Gellman, Acc. Chem. Res. 41:1399-408 (2008); Seebach & Gardiner, Acc. Chem. Res. 41:1366-75 (2008); Cummings & Hamilton, Curr. Opin. Chem. Biol. 14:341-46 (2010)). A hydrogen bond surrogate (HBS) approach that reproduces the conformation of proteinaceous α-helices in short peptide sequences was developed previously (Patgiri et al., Acc. Chem. Res. 41:1289-300 (2008)). HBS α-helices feature a hydrocarbon linkage in place of an N-terminal i→i+4 hydrogen bond, which nucleates the desired helical conformation in the appended peptide chain (Chapman et al., Biochemistry 47:4189-95 (2008); Wang et al., Org. Biomolec. Chem. 4:4074-81 (2006)). One of the key advantages of the HBS approach is that all amino acid side-chains remain available for molecular recognition. HBS helices have been shown to bind chosen protein targets in cell free and cell-based assays (Patgiri et al., Nature Chem. Biol. 7:585-87 (2011); Henchey et al., J. Am. Chem. Soc. 132:941-43 (2010); Henchey et al., ChemBiochem 11:2104-07 (2010); Wang et al., Angew. Chem. Int'l Ed. 47:1879-82 (2008)).

The hydrocarbon linkage of an HBS peptidomimetic is installed using a ring closing olefin metathesis reaction between an N-terminal 4-pentenoic acid residue, formally occupying the i^{th} position on the helix, and an i+4 N-allyl group (Patgiri et al., Org. Biomol. Chem. 8:1773-76 (2010); Chapman & Arora, Org. Lett. 8:5825-28 (2006); Dimartino et al., Org. Lett. 7:2389-92 (2005)). The optimized metathesis conditions require high reaction temperatures and catalyst loadings, which can result in product mixtures that are difficult to purify. Purification difficulties have restricted the use of HBS helices.

### SUMMARY OF THE INVENTION

The inventive embodiments herein are directed to overcoming these and other deficiencies. The inventive embodiments provided in this Summary of the Invention are meant to be illustrative only and to provide an overview of selected inventive embodiments disclosed herein. The Summary of the Invention, being illustrative and selective, does not limit the scope of any claim, does not provide the entire scope of inventive embodiments disclosed or contemplated herein, and should not be construed as limiting or constraining the scope of this disclosure or any claimed inventive embodiment.

Provided herein are peptidomimetics, their salts (and compositions containing at least one of these), having a stable, internally constrained protein secondary structure containing a thioether-, ether-, or alkylamine-linked hydrogen bond surrogate.

Provided herein, unless otherwise indicated, is a compound of Formula I or its salt (and compositions containing at least one of these):

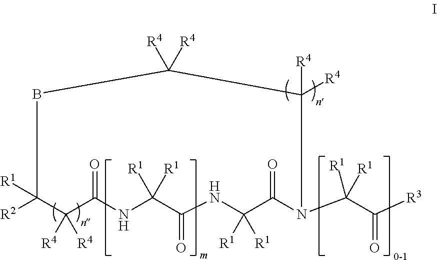

wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

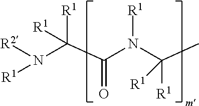

wherein:
   R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m′ is zero or any number; for example, m′ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
R³ is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

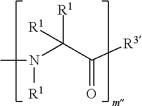

wherein:
   R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m″ is zero or any number; for example, m″ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
each R⁴ is independently hydrogen, an alkyl, an alkenyl, an alkynyl, a cycloalkyl, a heterocyclyl, an aryl, a heteroaryl, or an arylalkyl; and
m, n′, and n″ are each independently zero, one, two, three, or four, where the sum of m, n′, and n″ is from two to six, for example, 2, 3, 4, 5, or 6, or from 3 to 6, or from 4 to 6, or from 5 to 6, or from 2 to 5, or from 2 to 4, or from 2 to 3.

Provided herein, unless otherwise indicated, are compounds of Formula IIA or Formula IIB or their salts (and compositions containing at least one of these):

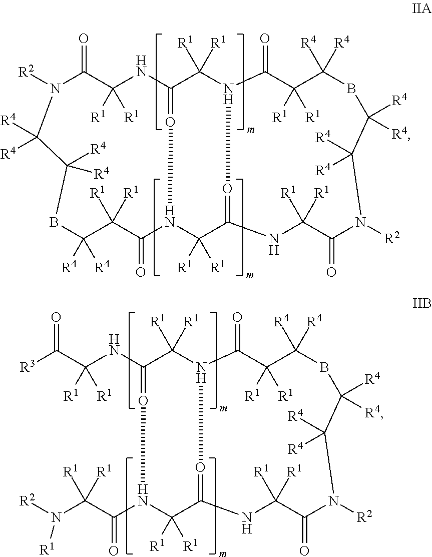

wherein:
each B is independently O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
each R² is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

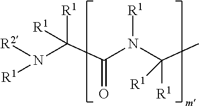

wherein:
   R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m′ is zero or any number; for example, m′ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
R³ is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

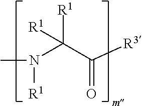

wherein:
   R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m″ is zero or any number; for example, m″ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m″ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
each R⁴ is independently hydrogen, an alkyl, an alkenyl, an alkynyl, a cycloalkyl, a heterocyclyl, an aryl, a heteroaryl, or an arylalkyl; and
each m is independently zero, one, two, three, or four.

Provided herein, unless otherwise indicated, is a method of preparing a compound of Formula IA or its salt:

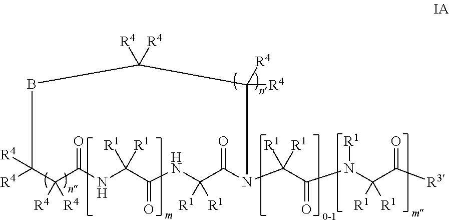

wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
each R⁴ is independently hydrogen, an alkyl, an alkenyl, an alkynyl, a cycloalkyl, a heterocyclyl, an aryl, a heteroaryl, or an arylalkyl;
   m″ is zero or any number; for example, m″ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m″ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
and
m, n′, and n″ are each independently zero, one, two, three, or four, where the sum of m, n′, and n″ is from two to six, for example, 2, 3, 4, 5, or 6, or from 3 to 6, or from 4 to 6, or from 5 to 6, or from 2 to 5, or from 2 to 4, or from 2 to 3.

Provided herein, unless otherwise indicated, is a method of preparing a compound of Formula IB or its salt:

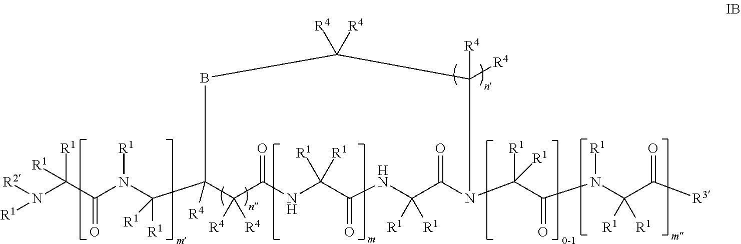

wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
each R⁴ is independently hydrogen, an alkyl, an alkenyl, an alkynyl, a cycloalkyl, a heterocyclyl, an aryl, a heteroaryl, or an arylalkyl;
   m′ and m″ are independently zero or any number; for example, m′ and m″ can each independently be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ and m″ can each independently range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
and
m, n′, and n″ are each independently zero, one, two, three, or four, where the sum of m, n′, and n″ is from two to six, for example, 2, 3, 4, 5, or 6, or from 3 to 6, or from 4 to 6, or from 5 to 6, or from 2 to 5, or from 2 to 4, or from 2 to 3; or a compound of Formula IC or its salt:

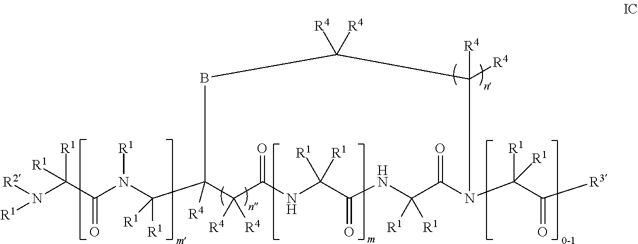

wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
each R⁴ is independently hydrogen, an alkyl, an alkenyl, an alkynyl, a cycloalkyl, a heterocyclyl, an aryl, a heteroaryl, or an arylalkyl;
   m′ is zero or any number; for example, m′ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
and
m, n′, and n″ are each independently zero, one, two, three, or four, where the sum of m, n′, and n″ is from two to six, for example, 2, 3, 4, 5, or 6, or from 3 to 6, or from 4 to 6, or from 5 to 6, or from 2 to 5, or from 2 to 4, or from 2 to 3.

Herein, unless otherwise indicated, is provided a method for promoting cell death. This method comprises, for example, contacting a cell with one or more compounds of Formula I or their salts that fully or partially inhibit p53/hDM2 interaction, under conditions effective for the one or more compounds or their salts to promote cell death. Herein, the method can, for example be an in vitro or an in vivo method.

Herein, unless otherwise indicated, is provided a facile and efficient synthesis of thioether-, ether-, and alkylamine-linked hydrogen bond surrogate peptide secondary structures and their salts. The traditional hydrocarbon-linked HBS helices have proven to be an exciting class of protein domain mimetics; however, their difficult synthesis has limited their usage. Facile synthesis of the thioether, ether, and alkylamine linkages allows one to bypass the ring-closing metathesis reaction—one of the key difficult reactions. It has been found that thioether-linked HBS (“teHBS”) helices compare favourably to carbon-linked HBS α-helices in conformational stability and protein targeting potential, and it is expected that ether- and alkylamine-linked HBS helices will as well.

Herein, unless otherwise indicated, is provided use of a compound herein, its salt, or a composition containing at least one of these, to cause or promote cell death.

Herein, unless otherwise indicated, is provided use of a compound herein or its salt to make a medicament for promoting cell death.

Herein, unless otherwise indicated, is provided a method of making a composition comprising any compound herein and/or its salt, comprising, for example, combining the compound herein or its salt with, for example, an excipient, or vehicle, to form the composition, which optionally can be a pharmaceutically acceptable composition.

### BRIEF DESCRIPTION OF THE DRAWINGS

FIG. 1 is a comparison of a canonical α-helix featuring an i→i+4 hydrogen bond with the hdyrocarbon linkage of an original HBS α-helix and the thioether linkage of a teHBS α-helix of the present invention.

FIG. 2 shows a βanti-parallel sheet (top) and β sheet conformations (middle (antiparallel β-hairpin) and bottom (antiparallel β-sheet macrocycle)) that can be made using the thioether-, ether-, or alkylamine-linked HBS approach of the present invention. Thioether bonds are shown by way of example.

FIG. 3 illustrates thioether formation during synthesis of thioether-stabilized α-helices. X=any leaving group; R, R¹=any amino acid side chain; Y=amide, ester, or carboxylic acid; shaded circles indicate a solid support. In FIG. 3A, N-terminal cyclization results in a 13-membered macrocycle. FIG. 3B shows C-terminal and mid-chain cyclization resulting in a 14-membered macrocycle.

FIG. 4 is a mass spectrum of teHBS 1 after HPLC purification. m/z=1534.

FIGS. 5A-B are reserve phase analytical HPLC traces for teHBS 1. FIG. 5A is the trace for the crude peptide. Mobile phase: 0.1% trifluoroacetic acid acetonitrile-water (gradient=5-95% over 20 minutes). FIG. 5B is the trace for the peptide after one round of purification. Mobile phase: in 0.1% trifluoroacetic acid acetonitrile-water (gradient=10-60% over 45 minutes).

FIG. 6 is the circular dichroism spectrum for teHBS 1. Double minima at 208 and 222 nm and a maximum near 190 nm are indicative of an α-helix. Percent helicity at each concentration was calculated to be 30%.

FIG. 7 is the saturation binding curve of Mdm2₂₅₋₁₁₇ with fl-p53.

FIG. 8 shows how thioether-, ether-, and alkylamine-linked HBS protein secondary structures can be synthesized through conjugate addition (Method A) or nucleophilic substitution (Method B) reactions (teHBS 1 is shown by way of example).

FIG. 9 is the CD spectra of teHBS 1 and HBS 2 in 10% trifluoroethanol in phosphate buffered saline.

FIG. 10 is the ¹H NMR spectrum of teHBS 1 in ACN-d₃/5% DMSO-d₆.

FIG. 11 is the ¹H NMR spectrum of teHBS 1 in DMSO-d₆.

FIGS. 12A-B are short range (FIG. 13A) and medium-range (FIG. 13B) NOE's observed for teHBS 1. FIG. 13C is the NOESY correlation chart for teHBS 1. The glycine-3 residue is N-alkylated. Filled rectangles indicate relative intensity of the NOE cross-peaks. Empty rectangles indicate NOE that could not be unambiguously assigned because of overlapping signals.

FIG. 13 is a graph of the teHBS 1 and HBS 2 binding affinities for Mdm2 as determined by a fluorescence-polarization assay.

### INCORPORATION BY REFERENCE

All patents, patent applications, and publications, including electronic publications, referenced herein are incorporated by reference as if each individual patent, patent application, or publication was specifically and individually indicated to be incorporated by reference.

### DETAILED DESCRIPTION OF THE INVENTION

The details of one or more inventive embodiments are set forth in the accompanying drawings, the claims, and in the description herein. Other features, objects, and advantages of inventive embodiments disclosed and contemplated herein will be apparent from the description, and drawings, and from the claims.

As used herein, the article “a” means one or more unless explicitly otherwise provided for.

As used herein unless otherwise indicated, terms such as “contain,” “containing,” “include,” “including,” and the like mean “comprising.”

As used herein unless otherwise indicated, the term “or” can be conjunctive or disjunctive.

As used herein unless otherwise indicated, any embodiment can be combined with any other embodiment.

As used herein unless otherwise indicated, some inventive embodiments herein contemplate numerical ranges. When ranges are present, the ranges include the range endpoints. Additionally, every subrange and value within the range is present as if explicitly written out.

In the event of a conflict between a term herein and a term from an incorporated-by-reference patent, patent application, or publication, the term herein controls.

Provided herein are peptidomimetics and their salts (and compositions containing at least one of these) having a stable, internally constrained protein secondary structure containing a thioether-, ether-, or alkylamine-linked hydrogen bond surrogate (HBS), and methods of making and using them.

Protein secondary structures can be defined by the hydrogen bonding patterns observed between the various main chain amide groups. Analyses of helix-coil transition in peptides emphasize the energetically demanding organization of three consecutive amino acids into the helical orientation as the slow step in helix formation (Qian & Schellman, J. Chem. Phys., 96:3987-3994 (1992); Lifson & Roig, J. Chem. Phys., 34:1963-1974 (1961); Zimm & Bragg, J. Chem. Phys., 31:526-535 (1959), which are hereby incorporated by reference in their entirety). Preorganization of these amino acid residues is expected to overwhelm the intrinsic nucleation propensities and initiate helix formation (Austin et al., J. Am. Chem. Soc., 119:6461-6472 (1997); Kemp et al., J. Org. Chem., 56:6672-6682 (1991)). In an α-helix, for example, a hydrogen bond between the C═O of the i^{th} amino acid residue and the NH of the i+4^{th} amino acid residue stabilizes and nucleates the helical structure (see Scheme 1 infra). Similar interactions stabilize and nucleate other helices, β-sheet/β-hairpins, and other peptide secondary structures.

To mimic the C═O—H—N hydrogen bond, the peptidomimetics herein and their salts can incorporate a covalent bond of the type C₁₋₅—B—C₁₋₅—N, as shown in Scheme 1.

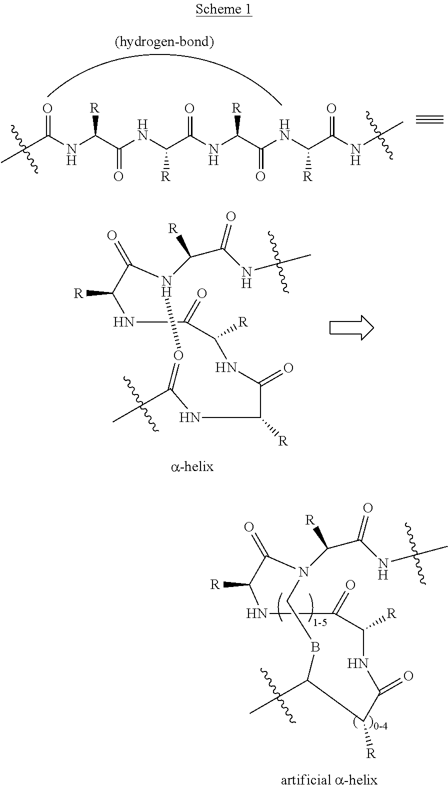

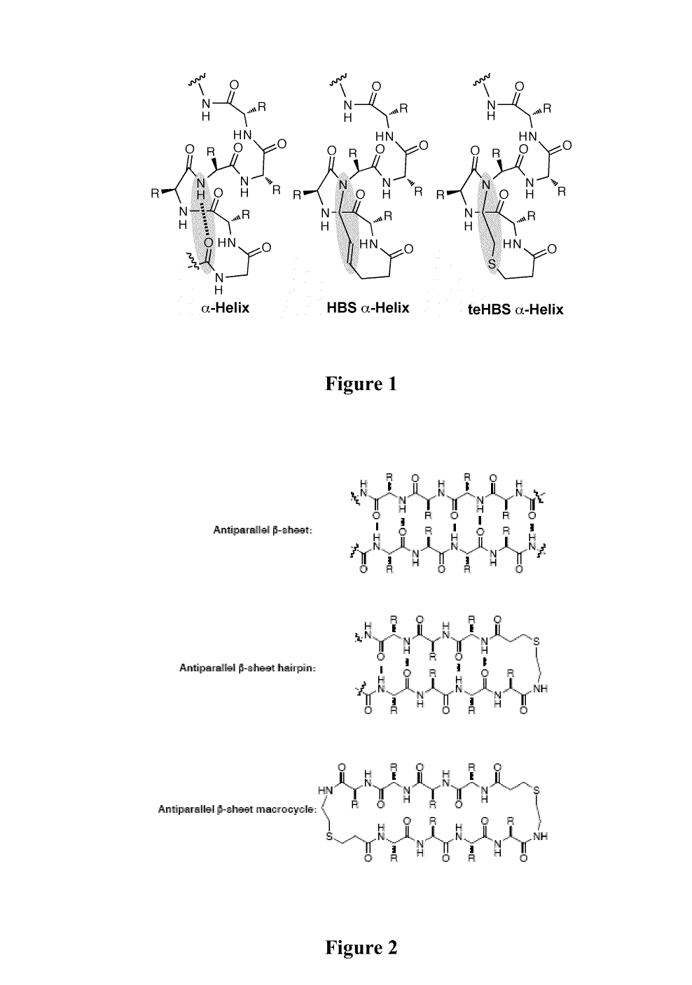

As shown in FIG. 1 (using an α-helix by way of example), as with hydrocarbon-based HBS peptidomimetics and their salts, the internal placement of the crosslink allows the development of protein secondary structures such that none of the exposed surfaces are blocked by the constraining element—i.e., placement of the crosslink on the inside of the protein secondary structure does not alter side-chain functionality nor block solvent-exposed molecular recognition surfaces of the molecule (see Sia et al., Proc. Nat'l Acad. Sci. USA 99:14664-14669 (2002)). Moreover, even very short peptides (i.e., peptides less than 10 amino acid residues, for example, peptides having about 9, or about 8, or about 7, or about 6, or about 5, or about 4, or about 3 residues) may be constrained into highly stable protein secondary structures.

Additionally, thioether-, ether-, and alkylamine-linked HBS peptidomimetics and therein salts herein can be easier to synthesize, and to synthesize in higher yield, than their hydrocarbon-linked HBS counterparts.

Protein secondary structures herein can include, without limitation, α-helices, 3₁₀-helices, pi helices, gramicidin helices, β-sheet macrocycles, and β-hairpins.

The stable, internally constrained protein secondary structures herein, unless otherwise indicated, can contain a thioether-, ether-, or alkylamine-linked HBS having the moiety

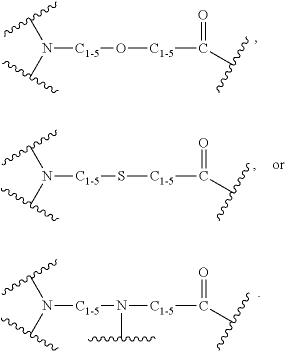 [SMILES: *C(=O)COCN(*)* ; *C(=O)CSCN(*)* ; *C(=O)CN(*)CN(*)*]

Herein unless otherwise indicated are provided compounds of Formula I or their salts (and compositions containing at least one of these):

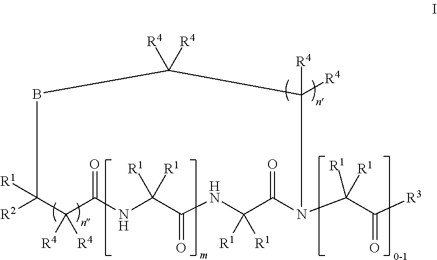

wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

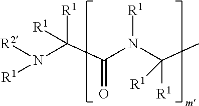

where:
   R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m′ is zero or any number; for example, m′ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
R³ is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

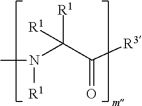

where:
   R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m″ is zero or any number; for example, m″ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
each R⁴ is independently hydrogen, an alkyl, an alkenyl, an alkynyl, a cycloalkyl, a heterocyclyl, an aryl, a heteroaryl, or an arylalkyl; and
m, n′, and n″ are each independently zero, one, two, three, or four, where the sum of m, n′, and n″ is from two to six, for example, 2, 3, 4, 5, or 6, or from 3 to 6, or from 4 to 6, or from 5 to 6, or from 2 to 5, or from 2 to 4, or from 2 to 3.

Herein unless otherwise indicated, amino acid side chains can be any amino acid side chain from natural or nonnatural amino acids, including from alpha amino acids, beta amino acids, gamma amino acids, L-amino acids, and D-amino acids. Amino acid side chains herein can include, for example unless otherwise indicated, side chains from arginine, histidine, lysine, aspartic acid, glutamic acid, serine, threonine, asparagine, glutamine, cysteine, selenocysteine, glycine, proline, alanine, valine, isoleucine, leucine, methonine, phenylalanine, tyrosine, or tryptophan.

As used herein, unless otherwise indicated, the term “alkyl” means an aliphatic hydrocarbon group which may be straight or branched having about 1 to about 6 carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl. Alkyl groups herein, unless otherwise indicated, can contain, for example, from 1 to 6 carbon atoms, from 1 to 4 carbon atoms, from 1 to 3 carbon atoms, from 1 to 3 carbon atoms, from 2 to 6 carbon atoms, from 3 to 6 carbon atoms, from 4 to 6 carbon atoms, from 5 to 6 carbon atoms, or 1, 2, 3, 4, 5, or 6, carbon atoms. The alkyl group may be substituted or unsubstituted.

The term “alkenyl” as used herein, unless otherwise indicated, means an aliphatic hydrocarbon group containing a carbon-carbon double bond and which may be straight or branched having about 2 to about 6 carbon atoms in the chain, for example about 2, about 3, about 4, about 5, or about 6 carbon atoms, or about 3 to about 6, about 4 to about 6, about 5 to about 6, about 2 to about 5, about 2 to about 4, or about 2 to about 3 carbon atoms. Preferred alkenyl groups have 2 to about 4 carbon atoms in the chain. Exemplary alkenyl groups include ethenyl, propenyl, n-butenyl, and i-butenyl. The alkenyl group may be substituted or unsubstituted.

The term “alkynyl” as used herein, unless otherwise indicated, means an aliphatic hydrocarbon group containing a carbon-carbon triple bond and which may be straight or branched having about 2 to about 6 carbon atoms in the chain, for example about 2, about 3, about 4, about 5, or about 6 carbon atoms, or about 3 to about 6, about 4 to about 6, about 5 to about 6, about 2 to about 5, about 2 to about 4, or about 2 to about 3 carbon atoms. Preferred alkynyl groups have 2 to about 4 carbon atoms in the chain. Exemplary alkynyl groups include ethynyl, propynyl, n-butynyl, 2-butynyl, 3-methylbutynyl, and n-pentynyl. The alkylyl group may be substituted or unsubstituted.

As used herein unless otherwise indicated, the term “cycloalkyl” refers to a non-aromatic saturated or unsaturated mono- or polycyclic ring system which may contain, for example, 3 to 6 carbon atoms, about 3, about 4, about 5, about 6, from about 4 to about 6, from about 5 to about 6, from about 3 to about 5, from about 3 to about 4, about 3, about 4, about 5, or about 6 carbon atoms and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane. The cycloalkyl group may be substituted or unsubstituted.

As used herein unless otherwise indicated, the term “heterocyclyl” can refer to a stable 3- to 18-membered, for example, 2, 3, 4, 5, 6, 7, 8, 9, 10, 11, 12, 13, 14, 15, 16, 17, 18, 3 to 18, 5 to 18, 6 to 18, 7 to 18, 8 to 18, 9 to 18, 10 to 18, 11 to 18, 12 to 18, 13 to 18, 14 to 18, 15 to 18, 16 to 18, 17 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4 membered ring system that comprises one or more carbon atoms and from one to five (e.g., 1, 2, 3, 4, or 5) heteroatoms each individually selected from the group consisting of nitrogen, oxygen, and sulfur. The heterocyclyl may be a monocyclic or a polycyclic ring system, which may include fused, bridged, or spiro ring systems; and the nitrogen, carbon, or sulfur atoms in the heterocyclyl may be optionally oxidized; the nitrogen atom may be optionally quaternized; and the ring may be partially or fully saturated. Representative monocyclic heterocyclyls include piperidine, piperazine, pyrimidine, morpholine, thiomorpholine, pyrrolidine, tetrahydrofuran, pyran, tetrahydropyran, oxetane, and the like. Representative polycyclic heterocyclyls include indole, isoindole, indolizine, quinoline, isoquinoline, purine, carbazole, dibenzofuran, chromene, xanthene, and the like. The heterocyclyl group may be substituted or unsubstituted.

As used herein unless otherwise indicated, the term “aryl” refers to an aromatic monocyclic or a polycyclic ring system containing from, for example, 6 to 19 carbon atoms, for example, 6, 7, 8, 9, 10, 11, 12, 13, 14, 15, 16, 17, 18, 19, from 8 to 19, from 10 to 19, from 12 to 19, from 14 to 19, from 16 to 19, from 6 to 17, from 6 to 15, from 6 to 13, from 6 to 11, or from 6 to 9 carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl. The aryl group may be substituted or unsubstituted.

As used herein unless otherwise indicated, “heteroaryl” refers to an aromatic ring system that comprises one or more carbon atoms and from one to five heteroatoms (e.g., 1, 2, 3, 4, or 5 hetero atoms) each individually selected from the group consisting of nitrogen, oxygen, and sulfur. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. The heteroaryl group may be substituted or unsubstituted.

As used herein unless otherwise indicated, the term “arylalkyl” refers to a moiety of the formula —R^{a}R^{b} where R^{a} is an alkyl or cycloalkyl as defined above and R^{b} is an aryl or heteroaryl as defined above. The arylalkyl group may be substituted or unsubstituted.

As used herein unless otherwise indicated, the term “acyl” means a moiety of formula R-carbonyl, where R is an alkyl, cycloalkyl, aryl, or heteroaryl as defined above. Exemplary acyl groups include formyl, acetyl, propanoyl, benzoyl, and propenoyl. The acyl group may be substituted or unsubstituted.

As used herein unless otherwise indicated, an amino acid can be any natural or non-natural amino acid. The amino acid can be, for example, arginine, histidine, lysine, aspartic acid, glutamic acid, serine, threonine, asparagine, glutamine, cysteine, selenocysteine, glycine, proline, alanine, valine, isoleucine, leucine, methonine, phenylalanine, tyrosine, or tryptophan.

A “peptide” as used herein, unless otherwise indicated, is any oligomer of two or more natural or non-natural amino acids, including alpha amino acids, beta amino acids, gamma amino acids, L-amino acids, D-amino acids, and combinations thereof. In preferred embodiments, the peptide is ˜5 to ˜30 (e.g., ˜5 to ˜10, ˜5 to ˜17, ˜10 to ˜17, ˜10 to ˜30, or ˜18 to ˜30) amino acids in length. The peptide can be, for example, 10-17 amino acids in length. The peptide can be, for example, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids in length.

A “tag” as used herein, unless otherwise indicated, includes any labeling moiety that facilitates the detection, quantitation, separation, and/or purification of the compounds herein or their salts. Suitable tags include purification tags, radioactive or fluorescent labels, and enzymatic tags.

Purification tags, such as poly-histidine (His₆₋), a glutathione-S-transferase (GST-), or maltose-binding protein (MBP-), can assist in compound purification or separation but can later be removed, i.e., cleaved from the compound following recovery. Protease-specific cleavage sites can be used to facilitate the removal of the purification tag. The desired product can be purified further to remove the cleaved purification tags.

Other suitable tags include radioactive labels, such as, ¹²⁵I, ¹³¹I, ¹¹¹In, or ⁹⁹TC. Methods of radiolabeling compounds are known in the art and described in U.S. Pat. No. 5,830,431 to Srinivasan et al. Radioactivity can be detected and quantified, for example, using a scintillation counter or autoradiography. Alternatively, the compound can be conjugated to a fluorescent tag. Suitable fluorescent tags can include, without limitation, chelates (europium chelates), fluorescein and its derivatives, rhodamine and its derivatives, dansyl, Lissamine, phycoerythrin, and Texas Red. The fluorescent labels can be conjugated to the compounds herein or their salts, for example, using techniques disclosed in CURRENT PROTOCOLS IN IMMUNOLOGY (Coligen et al. eds., 1991). Fluorescence can be detected and quantified, for example, using a fluorometer.

Enzymatic tags generally, for example, catalyze a chemical alteration of a chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Examples of suitable enzymatic tags include luciferases (e.g., firefly luciferase and bacterial luciferase; see e.g., U.S. Pat. No. 4,737,456 to Weng et al.), luciferin, 2,3-dihydrophthalazinediones, malate dehydrogenase, urease, peroxidases (e.g., horseradish peroxidase), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, microperoxidase, and the like. Techniques for conjugating enzymes to proteins and peptides are described in O'Sullivan et al., Methods for the Preparation of Enzyme—Antibody Conjugates for Use in Enzyme Immunoassay, in METHODS IN ENZYMOLOGY 147-66 (Langone et al. eds., 1981).

A targeting moiety herein, unless otherwise indicated, can function to (i) promote the cellular uptake of the compound, (ii) target the compound to a particular cell or tissue type (e.g., signaling peptide sequence), or (iii) target the compound to a specific sub-cellular localization after cellular uptake (e.g., transport peptide sequence).

To promote the cellular uptake of a compound or its salt herein, the targeting moiety may be a cell penetrating peptide (CPP). CPPs translocate across the plasma membrane of eukaryotic cells by a seemingly energy-independent pathway and have been used successfully for intracellular delivery of macromolecules, including antibodies, peptides, proteins, and nucleic acids, with molecular weights several times greater than their own. Several commonly used CPPs, including polyarginines, transportant, protamine, maurocalcine, and M918, are suitable targeting moieties for use in the present invention and are well known in the art (see Stewart et al., “Cell-Penetrating Peptides as Delivery Vehicles for Biology and Medicine,” Organic Biomolecular Chem. 6:2242-2255 (2008)). Additionally, methods of making CPP are described in U.S. Patent Application Publication No. 20080234183 to Hallbrink et al.

Another suitable targeting moiety useful for enhancing the cellular uptake of a compound or its salt here, for example, can be an “importation competent” signal peptide as disclosed by U.S. Pat. No. 6,043,339 to Lin et al. An importation competent signal peptide can be, for example generally about 10 to about 50 amino acid residues in length, for example, about 10, about 20, about 30, about 40 about 50, about 20 to about 50, about 30 to about 50, about 40 to about 50, about 10 to about 40, about 10 to about 30, about 10 to about 20, or about 20 to about 30 residues in length—typically hydrophobic residues—that render the compound or its salt capable of penetrating through the cell membrane from outside the cell to the interior of the cell. An exemplary importation competent signal peptide includes the signal peptide from Kaposi fibroblast growth factor (see U.S. Pat. No. 6,043,339 to Lin et al.). Other suitable peptide sequences can be selected from the SIGPEP database (see von Heijne G., “SIGPEP: A Sequence Database for Secretory Signal Peptides,” Protein Seq. Data Anal. 1(1):41-42 (1987)).

Another suitable targeting moiety herein, unless otherwise indicated, can be a signal peptide sequence capable of targeting the compounds of the present invention to a particular tissue or cell type. The signaling peptide can include at least a portion of a ligand binding protein. Suitable ligand binding proteins include high-affinity antibody fragments (e.g., Fab, Fab′ and F(ab′)₂, single-chain Fv antibody fragments), nanobodies or nanobody fragments, fluorobodies, or aptamers. Other ligand binding proteins include biotin-binding proteins, lipid-binding proteins, periplasmic binding proteins, lectins, serum albumins, enzymes, phosphate and sulfate binding proteins, immunophilins, metallothionein, or various other receptor proteins. For cell specific targeting, the signaling peptide is preferably a ligand binding domain of a cell specific membrane receptor. Thus, when the modified compound is delivered intravenously or otherwise introduced into blood or lymph, the compound will adsorb to the targeted cell, and the targeted cell will internalize the compound. For example, if the target cell is a cancer cell, the compound may be conjugated to an anti-C3B(I) antibody as disclosed by U.S. Pat. No. 6,572,856 to Taylor et al. Alternatively, the compound may be conjugated to an alphafeto protein receptor as disclosed by U.S. Pat. No. 6,514,685 to Moro, or to a monoclonal GAH antibody as disclosed by U.S. Pat. No. 5,837,845 to Hosokawa. For targeting a compound to a cardiac cell, the compound may be conjugated to an antibody recognizing elastin microfibril interfacer (EMILIN2) (Van Hoof et al., “Identification of Cell Surface for Antibody-Based Selection of Human Embryonic Stem Cell-Derived Cardiomyocytes,” J Proteom Res 9:1610-18 (2010)), cardiac troponin I, connexin-43, or any cardiac cell-surface membrane receptor that is known in the art. For targeting a compound to a hepatic cell, the signaling peptide may include a ligand domain specific to the hepatocyte-specific asialoglycoprotein receptor. Methods of preparing such chimeric proteins and peptides are described in U.S. Pat. No. 5,817,789 to Heartlein et al.

Another suitable targeting moiety herein, unless otherwise indicated, is a transport peptide that directs intracellular compartmentalization of the compound once it is internalized by a target cell or tissue. For transport to the endoplasmic reticulum (ER), for example, the compound can be conjugated to an ER transport peptide sequence. A number of such signal peptides are known in the art, including the signal peptide MMSFVSLLLVGILFYATEAEQLTKCEVFQ (SEQ ID NO: 1). Other suitable ER signal peptides include the N-terminus endoplasmic reticulum targeting sequence of the enzyme 17β-hydroxysteroid dehydrogenase type 11 (Horiguchi et al., “Identification and Characterization of the ER/Lipid Droplet-Targeting Sequence in 17β-hydroxysteroid Dehydrogenase Type 11,” Arch. Biochem. Biophys. 479(2):121-30 (2008)), or any of the ER signaling peptides (including the nucleic acid sequences encoding the ER signal peptides) disclosed in U.S. Patent Application Publication No. 20080250515 to Reed et al. Additionally, the compounds or their salts herein can contain, unless otherwise indicated, an ER retention signal, such as the retention signal KEDL (SEQ ID NO: 2). Methods of modifying the compounds herein or their salts to incorporate transport peptides for localization of the compounds to the ER can be carried out as described in U.S. Patent Application Publication No. 20080250515 to Reed et al.

For transport to the nucleus herein unless otherwise indicated, the compounds herein and their salts can, for example, include a nuclear localization transport signal. Suitable nuclear transport peptide sequences are known in the art, including the nuclear transport peptide PPKKKRKV (SEQ ID NO:3). Other nuclear localization transport signals include, for example, the nuclear localization sequence of acidic fibroblast growth factor and the nuclear localization sequence of the transcription factor NF-KB p50 as disclosed by U.S. Pat. No. 6,043,339 to Lin et al. Other nuclear localization peptide sequences known in the art are also suitable for use in the compounds and their salts herein.

Suitable transport peptide sequences for targeting to the mitochondria include, for example, MLSLRQSIRFFKPATRTLCSSRYLL (SEQ ID NO: 4). Other suitable transport peptide sequences suitable for selectively targeting compounds and their salts herein to the mitochondria are disclosed in U.S. Patent Application Publication No. 20070161544 to Wipf.

As will be apparent to those of ordinary skill in the art, when R² and/or R³ are a moiety of the recited formulae, the overall size of the compounds of Formula I and their salt, unless otherwise indicated, can be adjusted by varying the values of m′ and/or m″, which are independently zero or any number. Typically, m′ and m″ are independently from zero to about thirty (e.g., 0 to ˜18, 0 to ˜10, 0 to ˜5, ˜5 to ˜30, ˜5 to ˜18, ˜5 to ˜10, ˜8 to ˜30, ˜8 to ˜18, ˜8 to ˜10, ˜10 to ˜18, or ˜10 to ˜30). Herein unless otherwise indicated, m′ and m″ can be independently 4-10. Herein unless otherwise indicated, m′ and m″ can be independently 5-6.

As will be apparent to the skilled artisan, compounds of Formula I and their salts, unless otherwise indicated, can include a diverse range of helical conformation, which depends on the values of m, n′, and n″. These helical conformations include 3₁₀-helices (e.g., m=0 and n′+n″=2), α-helices (e.g., m=1 and n′+n″=2), π-helices (e.g., m=2 and n′+n″=2), and gramicidin helices (e.g., m=4 and n′+n″=2). In a preferred embodiment, the number of atoms in the backbone of the helical macrocycle can be 12-15, or 13 or 14.

Herein unless otherwise indicated, the compound of Formula I or its salt can be a compound of Formula IA (i.e., a helix cyclized at the N-terminal) or its salt, Formula IB (i.e., a helix cyclized mid-peptide) or its salt, or Formula IC (i.e., a helix cyclized at the C-terminal) or its salt:

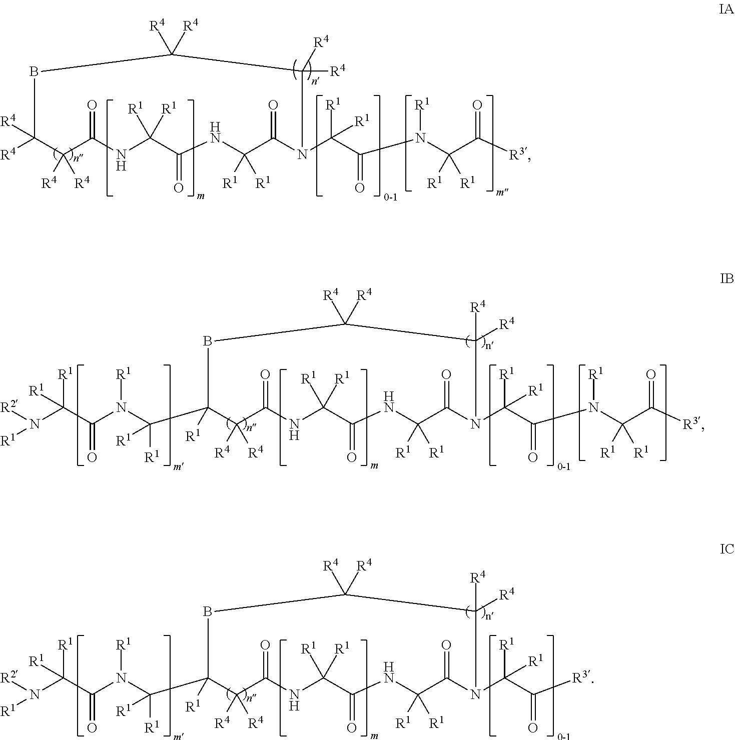

Herein unless otherwise indicated, are provided compounds of Formula IIA (i.e., a β-sheet macrocycle) or Formula IIB (i.e., a β-hairpin) or their salts:

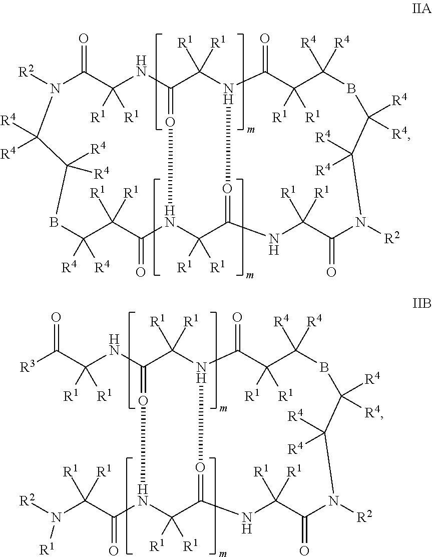

wherein:
each B is independently O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
each R² is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

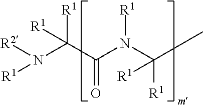

wherein:
   R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m′ is zero or any number; for example, m′ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
R³ is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula

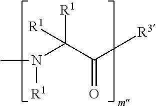

wherein:
   R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ where R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ where each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
   m″ is zero or any number; for example, m″ can be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; for example, m′ can range, for example, from 0 to 40, from 0 to 30, from 0 to 20, from 0 to 10, from 0 to 5, from 5 to 40, from 10 to 40, from 20 to 40, from 30 to 40, from 5 to 35, from 10 to 30, or from 15 to 25;
each R⁴ is independently hydrogen, an alkyl, an alkenyl, an alkynyl, a cycloalkyl, a heterocyclyl, an aryl, a heteroaryl, or an arylalkyl; and
each m is independently zero, one, two, three, or four.

Herein unless otherwise indicated, compounds herein and their salts include those shown in FIG. 2. FIG. 2 shows exemplary β-sheets constrained via thioether bonds. As will be apparent to one of ordinary skill in the art, analogous compounds constrained via an ether bond or alkylamine bond are also contemplated.

The compounds herein and their salts, unless otherwise indicated, may be prepared in accordance with the methods described herein.

Herein, unless otherwise indicated, is provided a method of preparing a compound of Formula 1A or its salt. This method is set forth in Table 1 below.

TABLE 1
Exemplary Method for Producing Compounds of Formula IA.
STEP 1: PREPARE THE C-TERMINAL END
(optional-skip if m″ is zero)

| | |
|---|---|
| (i) | PG³—D—Y—LG³ + a PG³ deprotecting agent → H—D—Y—LG³ + |
| (ii) | PG¹—AA—LG¹ → PG¹—AA—D—Y—LG³ |
| (iii) | repeat (i), substituting the PG³ deprotecting agent with a PG¹ deprotecting agent, and |
| | (ii) until m″ is the desired number → PG¹—[AA]_{m″}—D—Y—LG³ 1 |

STEP 2: ADD THE HETEROATOM
Skip (ii) if —N~B is attached directly to Y

| | |
|---|---|
| (i) | 1 or PG³—D—Y—LG³ + a PG¹ or PG³ deprotecting agent → H—[AA]_{m″}—D—Y—LG³ + |
| (ii) | 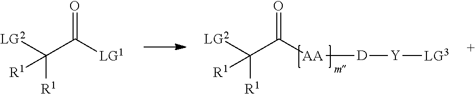 |
| (iii) | 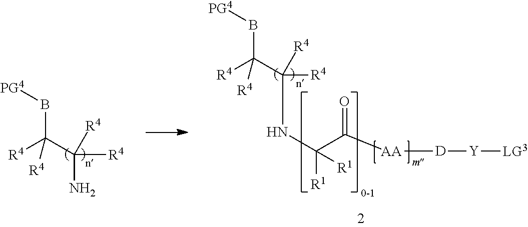 |

STEP 3: ADD RESIDUE(S)
Skip (ii)-(iv) if m is zero

| | |
|---|---|
| (i) | 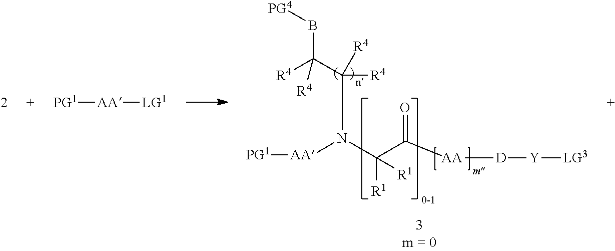 |
| (ii) | 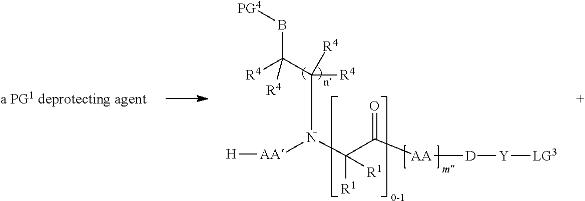 |
| (iii) | 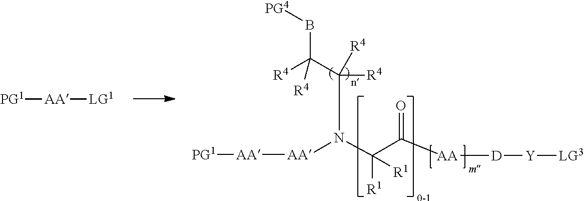 |
| (iv) | repeat (ii) and (iii) until m is the desired number → |
| | 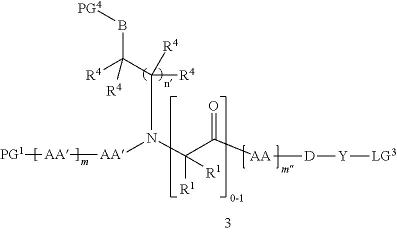 |

STEP 4: ADD THE ETHER PRECURSOR

| | |
|---|---|
| (i) | 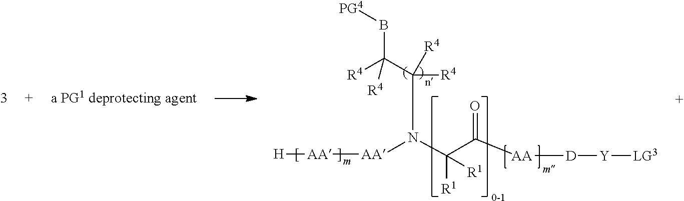 |
| (ii) | 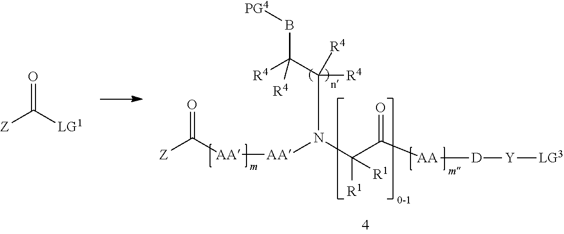 |

STEP 5: CYCLIZE

| | |
|---|---|
| (i) | 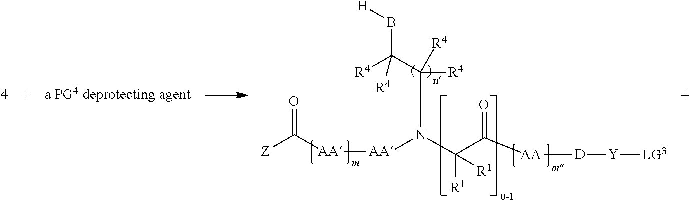 |
| (ii) | 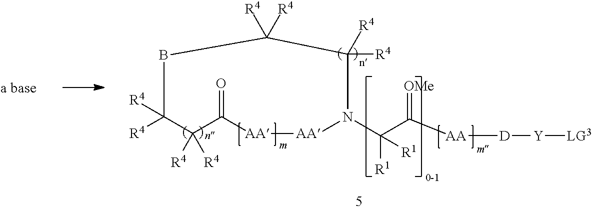 |

STEP 6: DEPROTECTION/CLEAVAGE
(i) and (ii) can happen in either order or simultaneously

| | |
|---|---|
| (i) | 5 + a PG² deprotecting agent^{†} + a cleaving agent^{‡} → |
| | 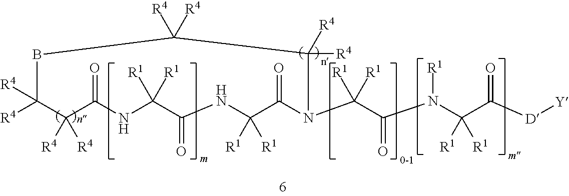 |
| | ^{†}If 5′ contains any PG²s |
| | ^{‡}If Y is a surface for solid phase synthesis |
| (ii) | convert —D′—Y′ to R^{3′} → |
| | 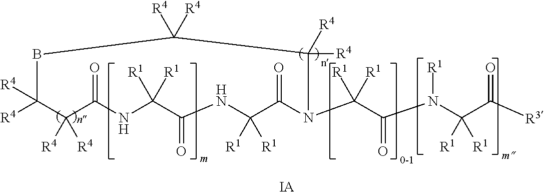 |

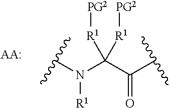

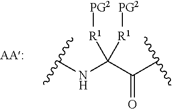

D: NR¹ or O
LG¹: a carboxyl activating group (e.g., dialkylcarbodiimide, hydroxybenzotriazole) or a halide (e.g., chloride, bromide)
LG²: any leaving group (e.g., halogen, tosylale, mesylate)
LG³: absent, a surface for solid phase synthesis, an alkyl/aryl ester, or an alkyl/aryl amide
R: an amino acid side chain
PG¹: a protecting group for the protection of an amine
PG²: absent or a protecting group for protection of the R¹ to which it is attached
PG³: if D is NR¹, a protecting group for the protection of an amine; if D is O, a protecting group for the protection of an alcohol
PG⁴: if B is O, a protecting group for the protection of an alcohol; if B is S, a protecting group for the protection of a thiol; if B is NR⁴, a protecting group for the protection of an amine
PG¹, PG², and PG³ are different
PG¹, PG², and PG⁴ are different
Y: H, alkyl, cycloalkyl, aryl, heteroaryl, arylalkyl, or a surface for solid phase synthesis
—D′—Y′: H if Y is a surface for solid phase synthesis; otherwise, —D—H or H
Z: CR¹₂═CR⁴— or X—CR¹₂—(CR⁴₂)_{n″}—
B, m, n′, n″, R¹, R², R³ and R⁴: as defined supra

Herein, unless otherwise indicated, is provided a method of preparing a compound of Formula IB or IC or their salts. This method is set forth in Table 2 below.

TABLE 2
Exemplary Method for Producing Compounds of Formula IB and IC.
STEP 1: PREPARE THE C-TERMINAL END
(optional-skip if m″ is zero)

| | |
|---|---|
| (i) | PG³—D—Y—LG³ + a PG³ deprotecting agent → H—D—Y—LG³ + |
| (ii) | PG¹—AA—LG¹ → PG¹—AA—D—Y—LG³ |
| (iii) | repeat (i), substituting the PG³ deprotecting agent with a PG¹ deprotecting agent, and |
| | (ii) until m″ is the desired number → PG¹—[AA]_{m″}—D—Y—LG³ 1 |

STEP 2: ADD THE HETEROATOM
Skip (ii) if —N~B is attached directly to Y

| | |
|---|---|
| (i) | 1 or PG³—D—Y—LG³ + a PG¹ or PG³ deprotecting agent → H—[AA]_{m″}—D—Y—LG³ + |
| (ii) | 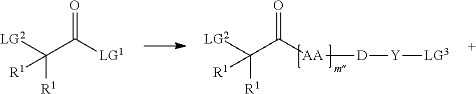 |
| (iii) | 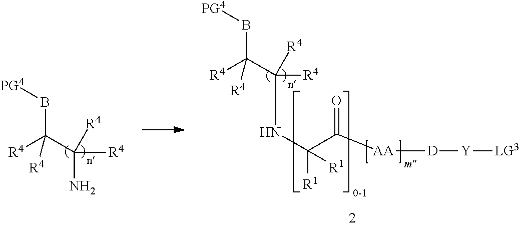 |

STEP 3: ADD RESIDUE(S)
Skip (ii)-(iv) if m is zero

| | |
|---|---|
| (i) | 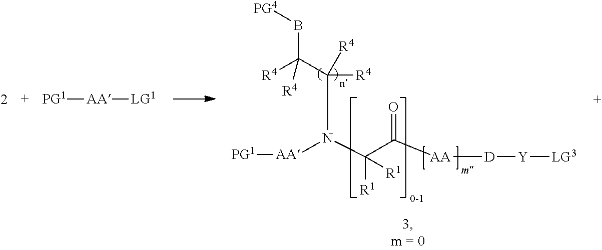 |
| (ii) | 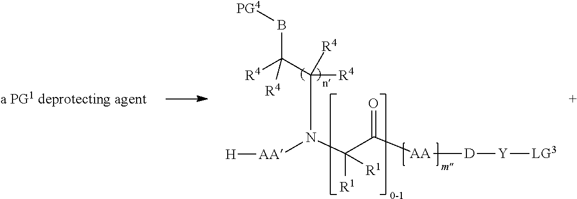 |
| (iii) | 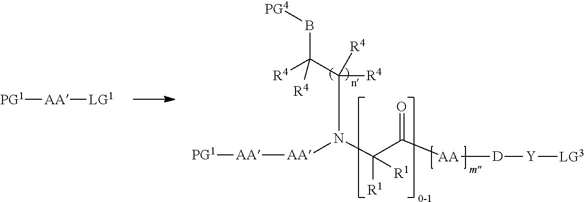 |
| (iv) | repeat (ii) and (iii) until m is the desired number → |
| | 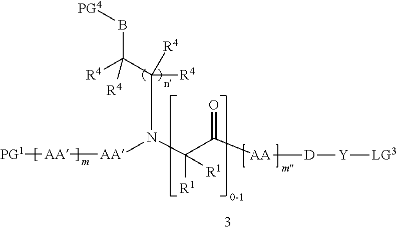 |

STEP 4: ADD A DIPEPTIDE ANALOG

| | |
|---|---|
| (i) | 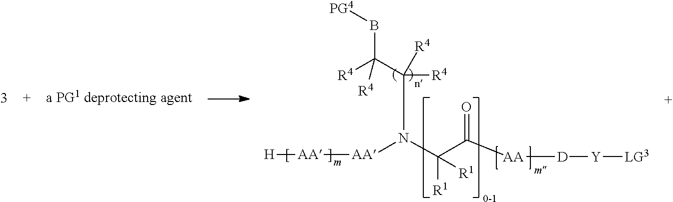 |
| (ii) | 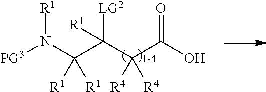 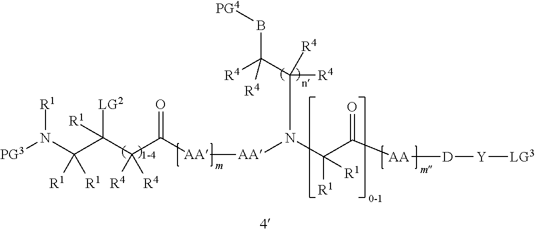 |

STEP 5: CYCLIZE

| | |
|---|---|
| (i) | 4′ + a PG⁴ deprotecting agent → |
| | 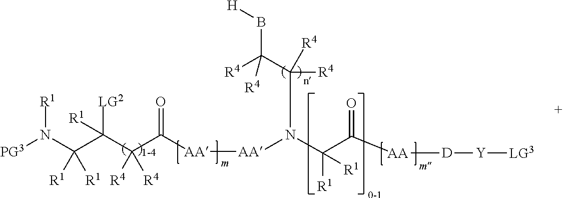 |
| (ii) | a base → |
| | 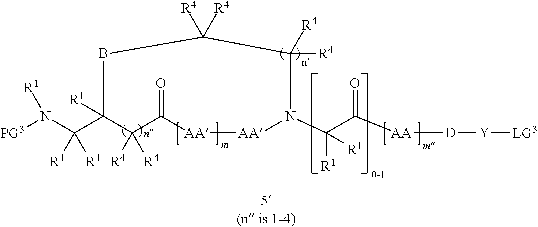 |

STEP 6: PREPARE THE N-TERMINAL END
(optional-skip if m′ is zero)
Skip (iii)-(v) if m′ is one

| | |
|---|---|
| (i) | 5′ + a PG³ deprotecting agent → |
| | 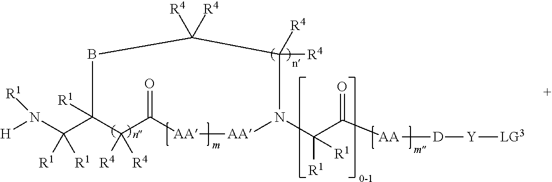 |
| (ii) | PG¹—AA—LG¹ → |
| | 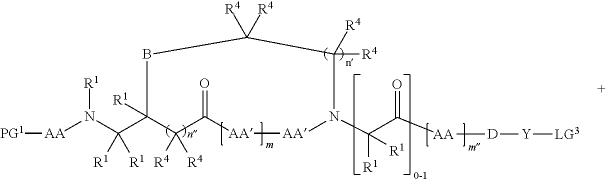 |
| (iii) | a PG¹ deprotecting agent → |
| | 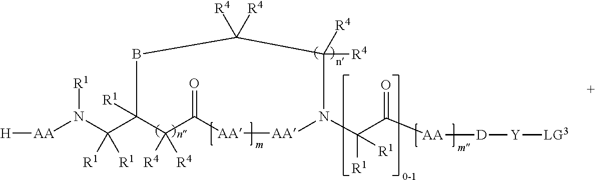 |
| (iv) | PG¹—AA—LG¹ → |
| | 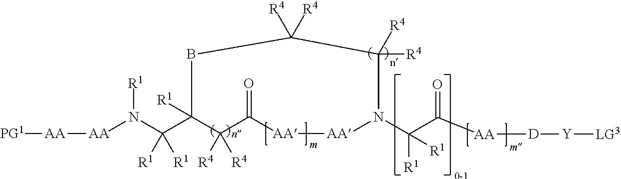 |
| (v) | repeat (iii) and (iv) until m′ is the desired number → |
| | 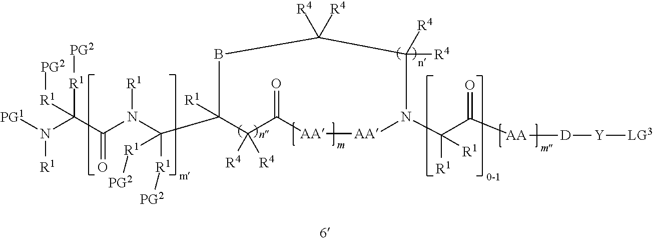 |

STEP 7: DEPROTECTION/CLEAVAGE
(i) and (ii) can happen in either order or simultaneously

| | |
|---|---|
| (i) | 5′ or 6′ + PG³ or PG¹ deprotecting agent + a PG² deprotecting agent^{†} + a cleaving agent^{‡} → |
| | 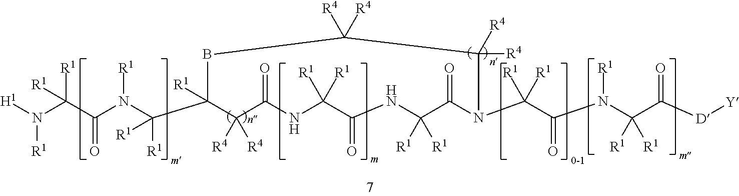 |
| | ^{†}If 5′ or 6′ contain any PG²s |
| | ^{‡}If Y is a surface for solid phase synthesis |
| (ii) | convert H¹ to R^{2′} and/or —D′—Y′ to R^{3′} → |
| | 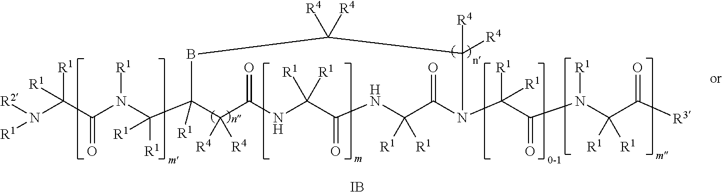 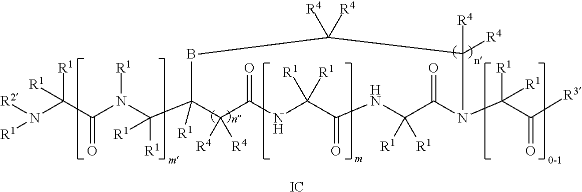 |

AA, AA′, B, D, LG¹, LG², LG³, m, n′, n″, R, R¹, R², R³, R⁴, PG¹, PG², PG³, PG⁴, Y, —D′—Y′, and Z: as defined supra

Herein unless otherwise indicated, leaving groups can be displaced as stable species taking with them the bonding electrons, resulting in coupling of one compound to another. Leaving groups that are suitable in the methods herein are well known in the art and include, without limitation, those employed in standard solution or solid phase peptide synthesis. Leaving groups herein can be, for example, tosylated or mesylated alcohols, Br, I, or Cl.

Protecting groups herein, unless otherwise indicated, function primarily to protect or mask the reactivity of functional groups. Protecting groups that are suitable for the protection of an amine group are well known in the art, including without limitation, carbamates, amides, N-alkyl and N-aryl amines, imine derivatives, enamine derivatives, and N-hetero atom derivatives as described by THEODORA W. GREENE & PETER G. M. WUTS, PROTECTIVE GROUPS IN ORGANIC SYNTHESIS 494-615 (1999). Suitable protecting groups herein, unless otherwise indicated, can include, e.g., tert-butyloxycarbonyl (“Boc”), 9-fluorenylmethyloxycarbonyl (“Fmoc”), carbobenzyloxy (“Cbz”), and trityl. Protecting groups that are suitable for the protection of an alcohol are also well known in the art. Suitable alcohol protecting groups include, without limitation, silyl ethers, esters, and alkyl/aryl ethers. Protecting groups that are suitable for the protection of a thiol group are also well known in the art. Suitable thiol protecting groups include, without limitation, aryl/alkyl thio ethers and disulfides. As will be apparent to those of ordinary skill in the art, amino acid side chains of Asn, Asp, Gln, Glu, Cys, Ser, His, Lys, Arg, Trp, or Thr will typically need to be, but need not always be, protected while carrying out the methods described herein. Protecting groups that are suitable for protecting these amino acid side chains are also well known in the art. Methods of protecting and deprotecting functional groups vary depending on the chosen protecting group; however, these methods are well known in the art and described in THEODORA W. GREENE & PETER G. M. WUTS, PROTECTIVE GROUPS IN ORGANIC SYNTHESIS 372-450 and 494-615 (1999).

The methods herein, unless otherwise indicated, may be carried out in solution and/or on a surface for solid phase synthesis. Suitable surfaces for solid phase synthesis include, for example, particles, strands, precipitates, gels, sheets, tubing, spheres, containers, capillaries, pads, slices, films, plates, slides, discs, membranes, etc. These surfaces can be made from a wide variety of materials, including polymers, plastics, ceramics, polysaccharides, silica or silica-based materials, carbon, metals, inorganic glasses, membranes, or composites thereof. The substrate is preferably flat but may take on a variety of alternative surface configurations. Suitable surfaces include, without limitation, resins, polymer films (e.g., cellulose, nitrocellulose, acrylamide), inorganic membranes (e.g., aluminum oxide, zirconium oxide), ceramic membranes, artificial membranes, gold surfaces, silyl surfaces, and carbon surfaces (e.g., carbon nanotubes, carbon buckyballs). Other surface materials will be readily apparent to those of ordinary skill in the art upon review of this disclosure.

### Synthesis of Thioether-Stabilized α-Helices

By way of example, a process for preparing peptidomimetics and their salts containing a HBS helix constrained via a thioether bond using Fmoc solid phase synthesis is described below and in Examples 1-11. As will be apparent to one of ordinary skill in the art, this process can be modified for preparing thioether-linked HBS helices using other synthetic approaches, as well as for preparing other thioether-linked HBS protein secondary structures, and ether-, and alkylamine-linked HBS protein secondary structures herein.

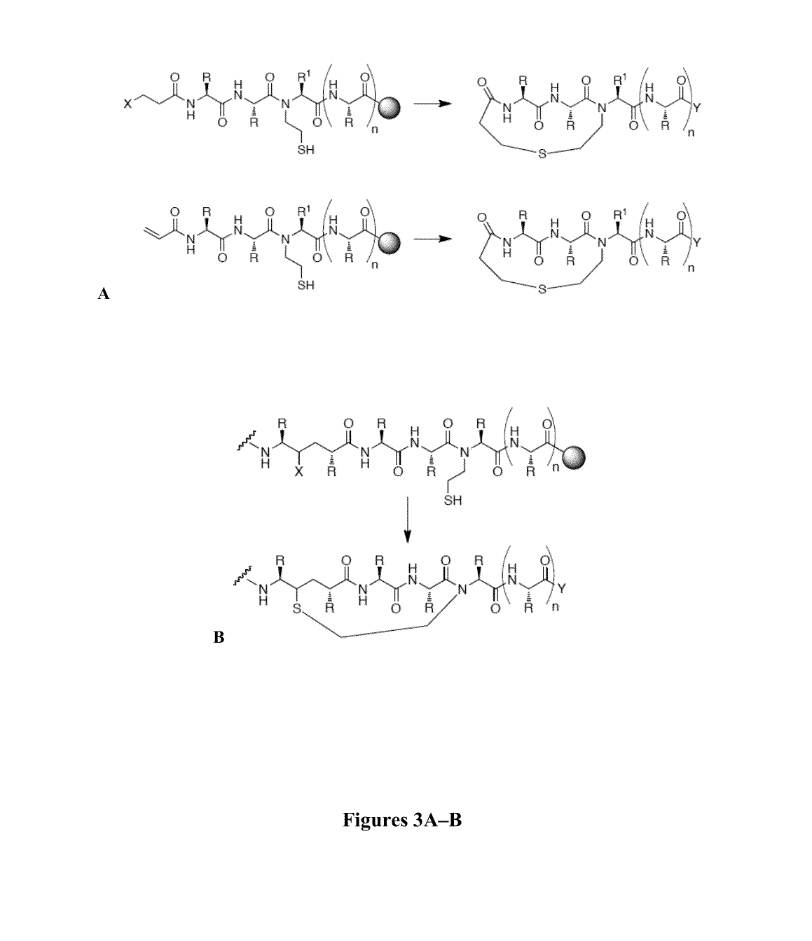

Thioether formation, for example, can be enabled by nucleophilic attack of a thiol at an electrophilic carbon center, as shown in FIG. 3. Cyclized peptides are expected to have improved binding affinities for protein targets and greater stability under physiological conditions, when compared to linear unconstrained peptide homologues. This method is amenable to Fmoc solid phase synthesis.

### N-Terminal Thioether Cyclized Peptides

When R¹ is any amino acid side chain besides glycine, it is predicted that introduction of the thiol can be performed, as shown in Scheme 2, via a Fukayama-Mitsunobu reaction with a protected thiol-containing alcohol (e.g. (S-monomethoxytrityl)-2-mercaptoethanol). When R¹ is glycine, an acetic acid derivative with a leaving group attached to the α-carbon (e.g. bromoacetic acid) can be coupled to the N-terminal amino acid residue followed by reaction with an excess of the protected thiol-containing primary amine (e.g. (S-monomethoxytrityl)-2-mercaptoethanol).

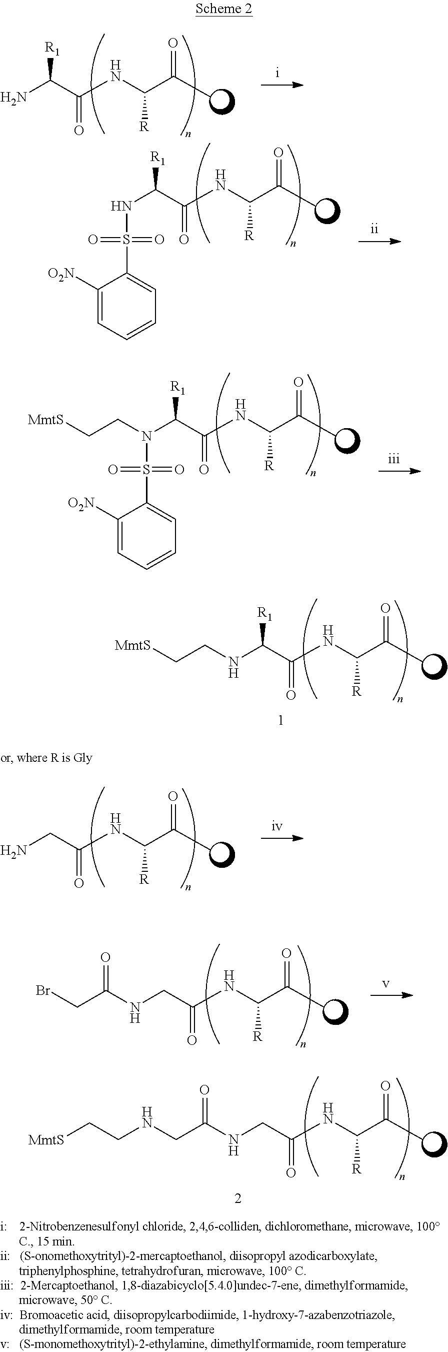

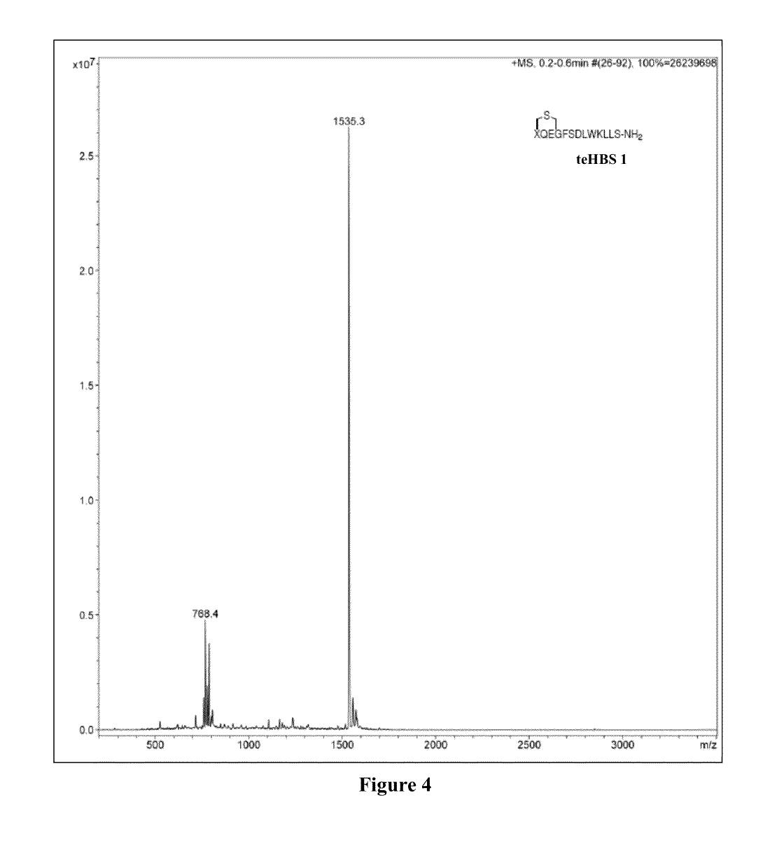

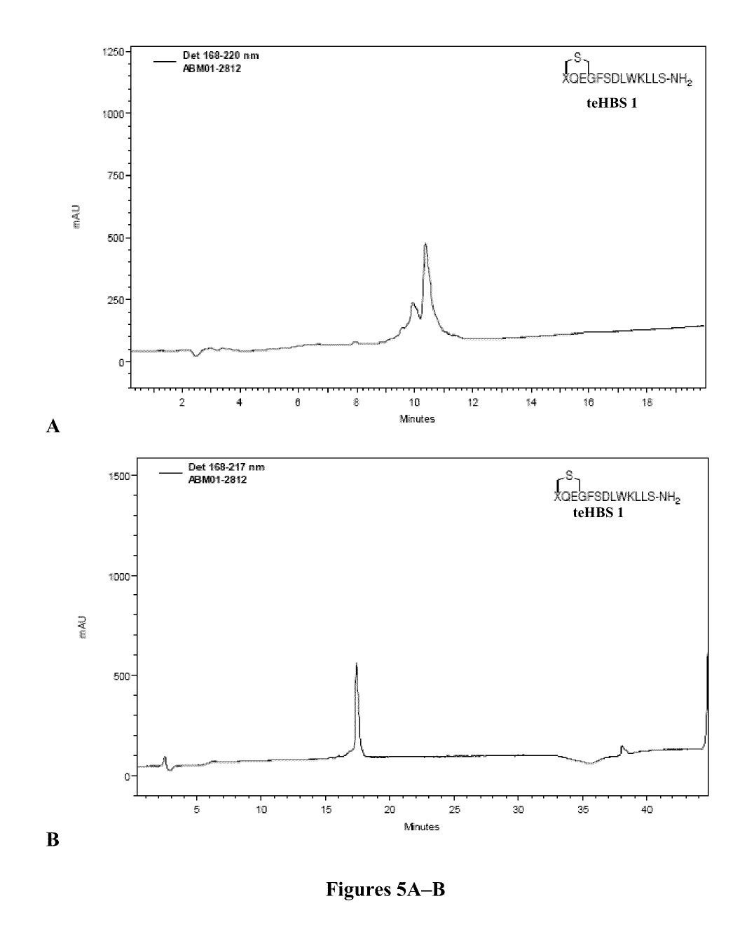

Fmoc-amino acid coupling to the secondary amine can be achieved by pre-activation with one or more peptide coupling reagents (e.g. triphosgene with a weak base, e.g. 2,4,6-collidine in tetrahydrofuran; diisopropylcarbodiimide and 1-hydroxy-7-azabenzotriazole), followed by microwave irradiation, as shown in Scheme 3. The terminal electrophilic (e.g. 3-bromopropionic acid) residue is appended using one or more peptide coupling reagents (e.g. diisopropylcarbodiimide and 1-hydroxy-7-azabenzotriazole) at room temperature. Selective removal of the protecting group (e.g. monomethyoxytrityl) can be achieved with a deprotecting agent (e.g. dichloromethane: trifluoroacetic acid: triisopropylsilane (93:2:5)). Successful removal of the protecting group can be confirmed using an Ellman colorimetric test. Cyclization can be achieved by addition of a base (e.g. 1,8-diazabicyclo[5.4.0]undec-7-ene in dimethylformamide) followed by shaking at room temperature (e.g. for 15 minutes). A negative Ellman test and mass spectrum indicating conversion of the thiol group to thioether can be used to confirm completion of the cyclization reaction (see FIG. 4 for the mass spectrum of teHBS 1).

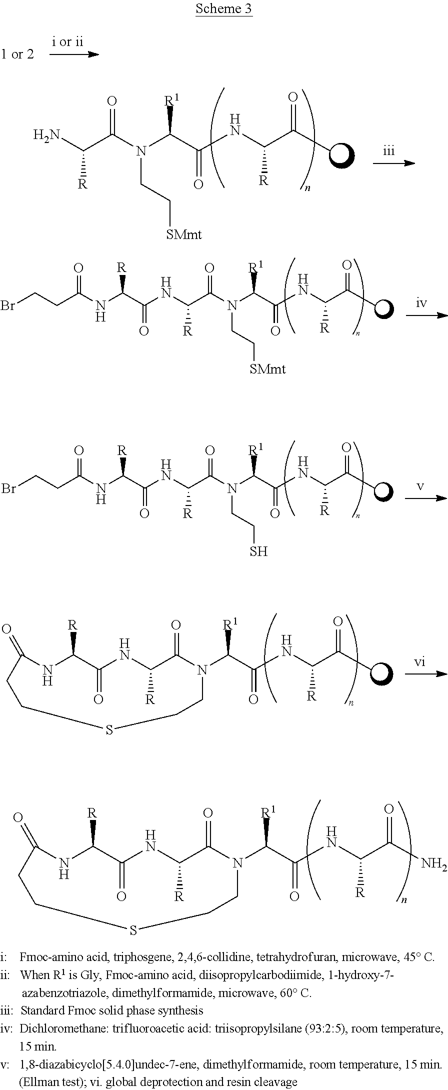

Global deprotection and cleavage from resin affords crude cyclized product, which can be further purified by reverse phase HPLC (see FIGS. 5A-B for example purification of teHBS 1).

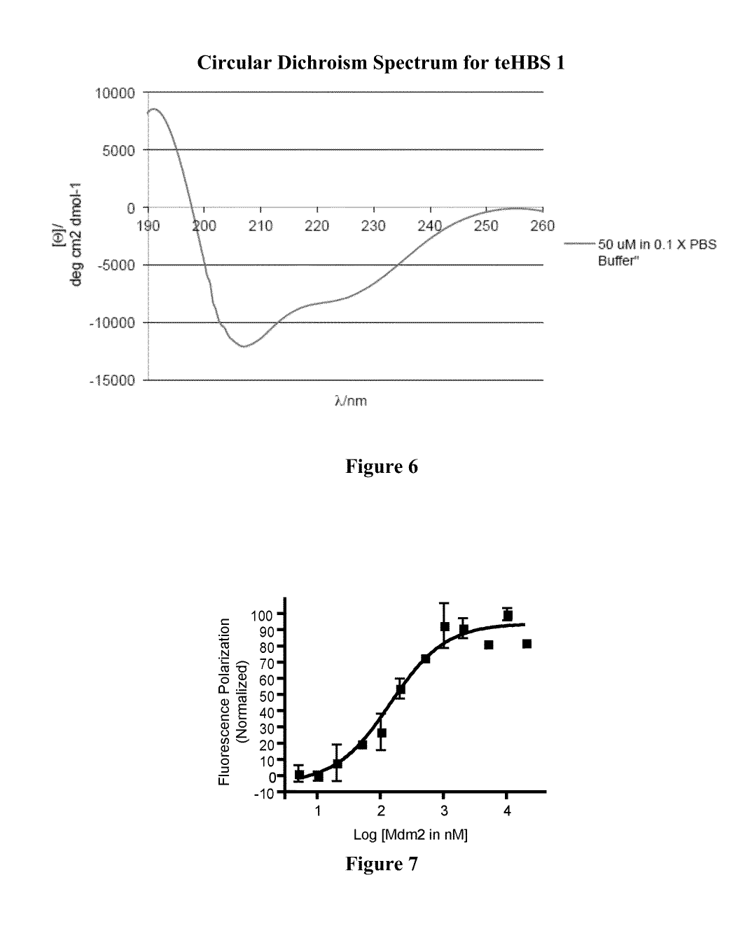

α-Helicity can be assessed by circular dichroism spectroscopy (CD). Minima at 208 and 222 nm and a maximum near 190 nm are indicative of canonical α-helices (see FIG. 6 for CD of teHBS 1).

### C-Terminal and Mid-Peptide Thioether Cyclized Peptides

The method for the introduction of C-terminal and mid-peptide thioether linkages is analogous to the introduction of an N-terminal thioether constraint, with a few differences. The electrophile is a dipeptide analog (e.g., 5) and must be pre-synthesized, for example as shown in Scheme 4. For synthesis of the dipeptide analog, an amino protecting group (e.g. Cbz) can be used for compatibility with strongly basic reagents that would be incompatible with Fmoc. The amino protecting group can be removed using standard protocols and peptide elongation achieved with standard Fmoc synthesis.

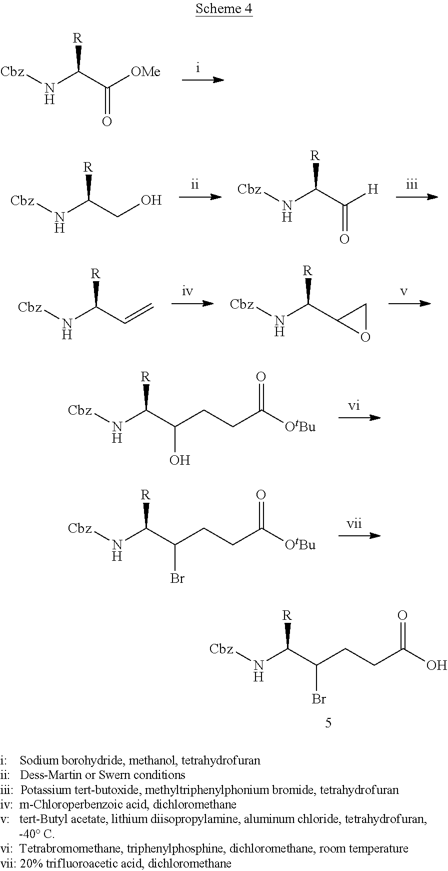

Due to the reactivity of the secondary alkyl halide, cyclization must be achieved before peptide elongation, as shown in Scheme 5.

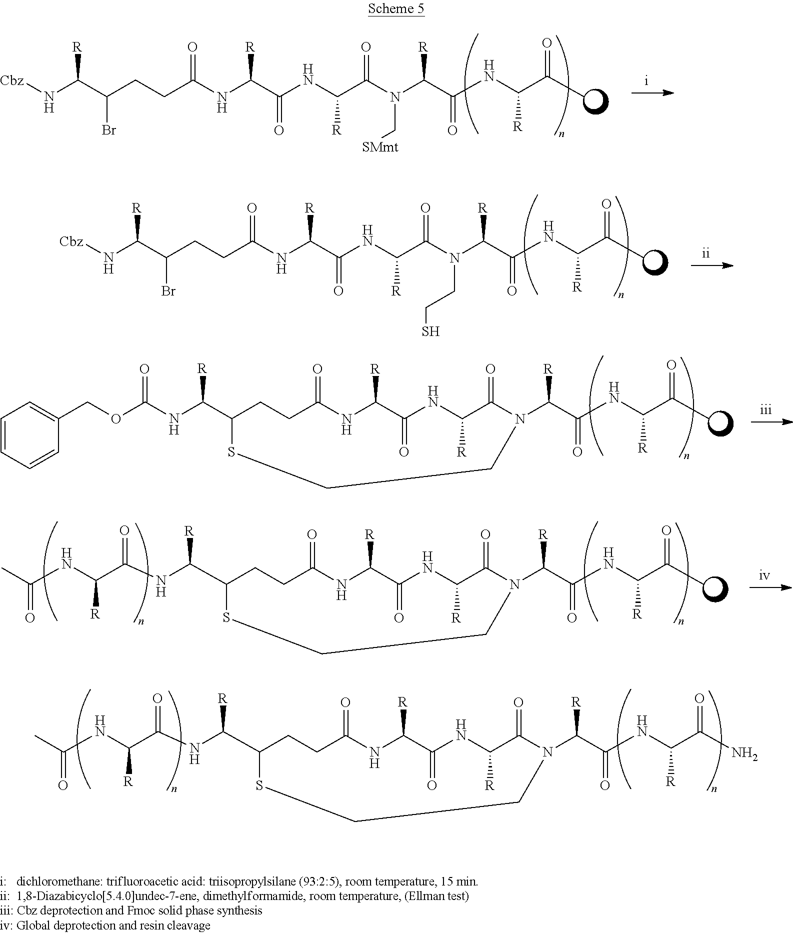

Provided herein, unless otherwise indicated, is a method for promoting cell death. This method can comprise, for example, contacting a cell with one or more compounds or their salts of Formula I (or compositions containing at least one of these) that fully or partially inhibit p53/hDM2, under conditions effective for the one or more compounds or their salts (or compositions containing at least one of these) to promote cell death.

Suitable p53/hDM2 inhibitors include teHBS 1.

The p53/hDM2 interaction is known to stop apoptosis and lead to uncontrolled growth (a characteristic of cancer). teHBS 1 mimics a portion of p53 protein that binds to hDM2, and is expected to block p53/hDM2 interaction and induce apoptotic activity in cancer cells (Chene, P, “Inhibiting the p53-MDM2 Interaction: An Important Target For Cancer Therapy,” Nat. Rev. Cancer 3:102-109 (2003); Chene et al., “Study of the Cytotoxic Effect of a Peptidic Inhibitor of the p53-HDN2 Interaction in Tumor Cells,” FEBS Lett. 529:293-297 (2002); Garcia-Echeverria et al., “Discovery of Potent Antagonists of the Interaction between Human Double Mminute 2 and Tumor Suppressor p53,” J. Medicinal Chemistry 43:3205-3208 (2000); Kritzer et al., “Helical β-Peptide Inhibitors of the p53-hDM2 Interaction,” J. Am. Chem. Soc. 126:9468-9469 (2004); Kussie et al., “Structure of the MDM2 Oncoprotein Bound to the p53 Tumor Suppressor Transactivation Domain,” Science 274: 948-953 (1996); Vassilev et al. “In Vivo Activation of the p53 Pathway by Small-molecule Antagonists of MDM2,” Science 303:844-848 (2004); Yin et al., “Terphenyl-based Helical Mimetics That Disrupt the p53/HDM2 Interaction,” Angew Chem. Int. Ed. 44:2704-2707 (2005)).

Contacting a cell with one or more compounds or their salts (or compositions containing at least one of these) herein, unless otherwise indicated, may be carried out in vitro or in vivo.

When contacting is carried out in vivo, contacting may comprise administering to a subject a compound or its salt herein (or a composition containing at least one of these). The subject may be a human. The subject may be in need thereof. The subject may be a non-human animal. The compounds herein, their salts, or compositions containing at least one of these, unless otherwise indicated, may be administered for example, orally, parenterally, for subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The, optionally active, compounds herein may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of, optionally active, compound. The percentage of the compound (optionally active) or its salt herein in these compositions may, of course, be varied and may conveniently be from about 2% to about 60%, about 4%, about 5%, about 8%, about 10%, about 15%, about 20%, about 25%, about 30%, about 35%, about 40%, about 45%, about 50%, about 55%, or about 60% of the weight of the unit. The amount of (optionally active) compound or its salt in such therapeutically useful compositions is such that a suitable dosage will be obtained. Preferred compositions according to the present invention are prepared so that an oral dosage unit contains from about 1 to about 250 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg, of (optionally active) compound or its salt.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These compounds and their salts herein (and compositions containing at least one of these) may also be administered parenterally. Solutions or suspensions of these can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds and their salts herein may also be administered directly to the airways in the form of an aerosol. For use as aerosols, the compounds and their salts herein in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

When using this method to treat a subject, or a subject in need thereof, the above-mentioned modes and forms of administering can be used to contact the cell with the one or more compounds of Formula I or their salts or compositions containing at least one of these.

The inventive embodiments herein may be further illustrated by reference to the following Examples. While inventive embodiments have been shown and described herein, such embodiments are provided by way of example only. Numerous variations, changes, and substitutions will now occur to those skilled in the art without departing from the inventive disclosure herein. The following Examples are illustrative and should not be construed as limiting.

### EXAMPLES

### Example 1

### Synthesis of teHBS 1

Thioether-derived hydrogen bond surrogate peptidomimetic teHBS 1 was prepared according to Scheme 6, as described in Examples 2-11.

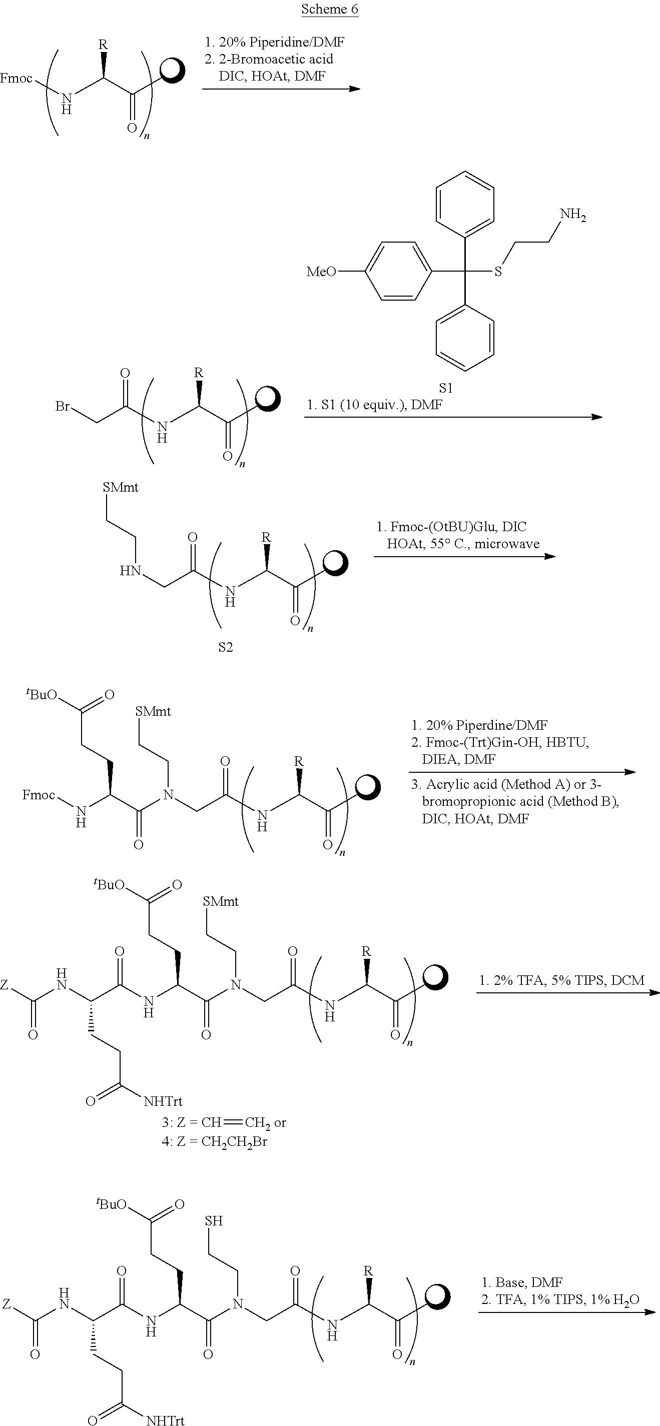

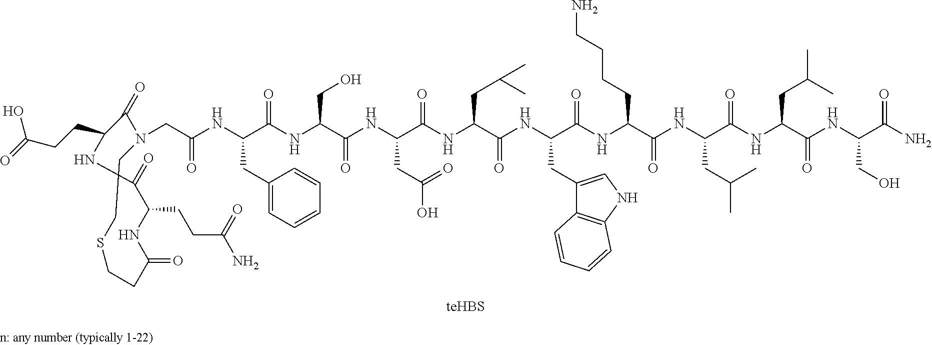 [SMILES: CC(C)C[C@H](NC(=O)[C@H](CC(C)C)NC(=O)[C@H](CCCCN)NC(=O)[C@H](Cc1c[nH]c2ccccc12)NC(=O)[C@H](CC(C)C)NC(=O)[C@H](CC(=O)O)NC(=O)[C@H](CO)NC(=O)[C@H](Cc1ccccc1)NC(=O)CN1CCSCCC(=O)N[C@@H](CCC(N)=O)C(=O)N[C@@H](CCC(=O)O)C1=O)C(=O)N[C@@H](CO)C(N)=O]

### Example 2

### General Materials and Methods

Commercial-grade reagents and solvents were used without further purification except as indicated. All Fmoc amino acids, peptide synthesis reagents, and Rink Amide MBHA resin were obtained from Novabiochem (San Diego, USA). All other reagents were obtained from Sigma-Aldrich (St. Louis, USA). Reversed-phase HPLC experiments were conducted with 4.6×150 mm (analytical scale) or 21.4×150 mm (preparative scale) Waters C18 Sunfire columns using a Beckman Coulter HPLC equipped with a System Gold 168 Diode array detector. The typical flow rates for analytical and preparative HPLC were 1 mL/min and 8 mL/min, respectively. In all cases, 0.1% aqueous trifluoroacetic acid and acetonitrile buffers were used. Proton and carbon NMR spectra of monomers were obtained on a Bruker AVANCE 400 MHz spectrometer. Proton NMR spectra of HBS peptides were recorded on a Bruker AVANCE 500 MHz spectrometer. High-resolution mass spectra (HRMS) were obtained on a LC/MSD TOF (Agilent Technologies). LCMS data were obtained on an Agilent 1100 series LC/MSD (XCT) electrospray trap.

### Example 3

### Synthesis of S-(4-Methoxytrityl)-2-aminoethanethiol

S-(4-methoxytrityl)-2-aminoethanethiol (“S1”(Riddoch et al., Bioconjugate Chem. 17:226-35 (2006))) was synthesized as follows. Cysteamine hydrochloride (1.75 g, 16.2 mmol) and 4-methoxytrityl chloride (5 g, 16 2 mmol) were dissolved in a mixture of DMF (25 mL) and dichloromethane (25 mL) and stirred at room temperature under an atmosphere of argon for 1 hour. The reaction mixture was concentrated in vacuo and diluted with water (150 mL) before extraction with diethyl ether (3×50 mL). The organic layers were combined, washed with brine (100 mL), dried over anhydrous magnesium sulfate, and evaporated to dryness to afford a colorless oil (5.5 g, 15.7 mmol, 97%). ¹H NMR (400 MHz; CDCl₃) δ 2.26 (2H, t, J 6.6 Hz), 2.53 (2H, t, J 6.6 Hz), 3.70 (3H, s), 6.72 (4H, m), 7.11 (1H, m), 7.16-7.25 (7H, m), 7.30-7.34 (2H, m). ¹³C NMR (100 MHz; CDCl₃) δ 36.24, 41.09, 55.23, 65.62, 113.11, 126.56, 127.85, 129.41, 130.69, 137.30, 145.53, 158.06.

### Example 4

### Preparation of the C-Terminal End

Knorr amide resin (0.69 mmol/g; 362 mg, 0.25 mmol) was swelled in DMF (5 mL) for 10 minutes prior to Fmoc group removal by treatment with 3 mL of 20% piperidine in NMP (5 minutes and then 20 minutes). The resin was then washed with DMF (3×5 mL), DCM (3×5 mL), and DMF (3×5 mL). The free amine was treated with pre-activated Fmoc-Ser(OtBu)-OH, which was prepared from Fmoc-Ser(OtBu)-OH (409 mg, 1.25 mmol), HBTU (474 mg, 1.25 mmol), and N,N-diisopropylethylamine (218 μL, 1.25 mmol) in DMF (3 mL). After 2 hours of shaking, the resin was washed with DMF (3×5 mL), DCM (3×5 mL), and DMF (3×5 mL). The Fmoc group was removed from the Fmoc-Ser(OtBu) functionalized resin using 20% piperidine in NMP (5 minutes and then 20 minutes) and the above procedure repeated for additional amino acid residues (Riddoch et al., Bioconjugate Chem. 17:226-35 (2006)).

### Example 5

### Addition of the Thiol

For inclusion of the ethane-2-thiol group, free amine was treated with pre-activated 2-bromoacetic acid, which was prepared from 2-bromoacetic acid (347 mg, 2.5 mmol), DIC (391 μL, 2.5 mmol), and HOAt (170 mg, 1.25 mmol) in DMF (3 mL). After 1 hour of shaking, the resin was washed with DMF (3×5 mL), DCM (3×5 mL), and DMF (3×5 mL). The bromoacetylgroup was treated with S1 (873 mg, 2.5 mmol) dissolved in DMF (3 mL). After 30 minutes of shaking, the resin was washed with DMF (3×5 mL), DCM (3×5 mL), and DMF (3×5 mL). Chloranil test was used to monitor the reaction progress.

### Example 6

### Addition of Amino Acid Residues

The secondary amine S2 (scheme 6) was treated with pre-activated Fmoc-Glu(OtBu)-OH and heated to 55° C. for 60 minutes under microwave conditions. Pre-activated Fmoc-Glu(OtBu)-OH was prepared from Fmoc-Glu(OtBu)-OH (532 mg, 1.25 mmol), DIC (196 μL, 1.25 mmol), and HOAt (85 mg, 0.63 mmol) in DMF (3 mL). The reaction was monitored using a chloranil test. The subsequent Fmoc-Gln(Trt) residue was incorporated using the method outlined above for coupling of Fmoc-Ser(OtBu)-OH to resin.

### Example 7

### Addition of the Electrophile

After removal of the Fmoc group, the free amine was treated with pre-activated acrylic acid for 3, which was prepared from acrylic acid (86 μL, 1.25 mmol), DIC (196 μL, 1.25 mmol), and HOAt (85 mg, 0.63 mmol) in DMF (3 mL). After 1 hour of shaking, the resin was washed with DMF (3×5 mL), DCM (3×5 mL), and DMF (3×5 mL). 3-Bromopropionic acid (191 mg, 1.25 mmol) was used in the place of acrylic acid for synthesis of 4.

### Example 8

### Selective S-4-Methoxytrityl Deprotection

Resin (0.25 mmol) was swelled in DCM (3 mL) before treatment with 2% TFA and 5% TIPS in DCM (5 mL). After shaking for 15 minutes, the resin was washed with DCM (3×5 mL). This deprotection procedure was repeated (typically three times) until no yellow color persisted in the reaction solvent. The resin was then washed with DMF (3×5 mL), DCM (3×5 mL), and DMF (3×5 mL). An Ellman test was used to confirm the presence of free thiol (Ellman, Arch. Biochem. Biophys. 82:70-77 (1959); Badyal et al., Tetrahedron Lett. 42:8531-33 (2001)).

### Example 9

### Cyclization via Method A

The free thiol functionalized resin, 3, was swelled in DMF (3 mL) before addition of appropriate base and the reaction was monitored using an Ellman test under the conditions shown in Table 3. Reactions were carried out at 25° C.

TABLE 3
On-Resin Cyclization Conditions for
Michael Addition Reaction with 3.

| | | Reaction | Ellman Test |
|---|---|---|---|
| Base | Equivalents | Time (h) | for Thiols |
| Triethylamine | 5 | 16 | Weak positive |
| Diisopropylethylamine | 5 | 16 | Weak positive |
| n-Butylamine | 5 | 16 | Negative |
| DBU | 5 | 12 | Negative |

### Example 10

### Cyclization via Method B

Free thiol functionalized resin, 4, was swelled in DMF (3 mL) before addition of appropriate base and the reaction was monitored using an Ellman test under the conditions shown in Table 4. Reactions were carried out at 25° C.

TABLE 4
On-Resin Cyclization Conditions for Substitution Reaction with 4.

| | | Reaction | | Ellman Test |
|---|---|---|---|---|
| Base | Equivalents | Time | | for Thiols |
| Triethylamine | 5 | 30 | min | Weak positive |
| N,N-Diisopropylethylamine | 5 | 30 | min | Weak positive |
| n-Butylamine | 5 | 2 | h | Negative |
| DBU | 5 | 10 | min | Negative |

### Example 11

### General Deprotection and Cleavage

Dried resin bound cyclized peptide was suspended in 3.8 ml of cleavage cocktail (TFA/H₂O/TIPS; 95%/2.5%/2.5%; vol/vol/vol) and agitated gently for 2 hours. The cleavage mixture was filtered and the resin washed with TFA (2×1 ml). The resin was discarded and the filtrate concentrated using a rotary evaporator. Cold diethyl ether (5 mL) was added slowly to the concentrated filtrate and the resulting precipitate isolated by centrifugation (5,000 g for 5 min.). The supernatant was decanted and the precipitate washed with diethyl ether (2×5 mL), followed by isolation using centrifugation (5,000 g for 5 min.) after each wash. The remaining solid was dissolved in a mixture of 0.1% TFA in water (vol/vol) and acetonitrile. This crude peptide solution was frozen and lyophilized to afford crude cyclized peptide.

### Example 12

### Expression and Purification of Mdm2 Fusion Protein

Competent BL21 DE3 pLySS E. coli cells were transformed by heat-shocking the bacteria at 42° C. for 30 seconds in media containing a pET-14B vector containing a His₆-tagged Mdm2₂₅₋₁₁₇ fusion protein. Cells were grown on ampicillin-containing agar plates (50 mg/mL), and a single culture was used to inoculate a 100 mL overnight culture of LB media containing ampicillin (50 mg/mL) at 37° C. 500 mL of terrific broth (4 L flask) was seeded with 25 mL of overnight culture and incubated at 30° C. for 5 hours (UV abs=1,600 nm) before induction of protein expression with 0.4 mM IPTG. The flask was incubated at 30° C. for an additional 4.5 hours. The cells were harvested by centrifugation at 3700 g for 45 minutes and the supernatant was discarded. The cells were resuspended in 50 mL of binding buffer (5 mM NaH₂PO₄, 30 mM NaCl, 0.5 mM imidazole, and 0.2 mM BME, Roche® protease inhibitor cocktail, pH 7.9), and lysed by sonication in ice (8×15 seconds pulses over 30 minutes). The cells were again centrifuged at 3700 g for 40 minutes at 4° C., and the resulting supernatant containing the desired Mdm2 fusion protein was allowed to bind nickel beads with shaking at 4° C. for 2 hours. Protein was eluted from the beads with elution buffer (5 mM NaH₂PO₄, 30 mM NaCl, 25 mM imidazole, and 0.2 mM BME). Protein was concentrated using an Amicon® Ultra centifuge filter (3 kD cut-off) and characterized by SDS-PAGE analysis.

### Example 13

### Protein Binding Studies

The relative affinity of peptides for N-terminal His₆-tagged Mdm2₂₅₋₁₁₇ was determined using a fluorescence polarization-based competitive binding assay with fluorescein-labeled p53 peptide (fl-p53). The polarization experiments were performed with a DTX 880 Multimode Detector (Beckman) at 25° C., with excitation and emission wavelengths at 485 and 525 nm, respectively. All samples were prepared in 96 well plates in dialysis buffer with 0.1% pluronic F-68 (Sigma). The binding affinity (K_{D}) values reported for each peptide are the averages of three individual experiments, and were determined by fitting the experimental data to a sigmoidal dose-response nonlinear regression model on GraphPad Prism 4.0. The concentration of the Mdm2 protein was determined by UV absorbance at 280 nm.

Prior to the competition experiments, the affinity of the fl-p53 for Mdm2₂₅₋₁₁₇ was determined by monitoring polarization of the fluorescent probe upon binding Mdm2₂₅₋₁₁₇ (FIG. 7). Addition of an increasing concentration (0 nm to 4 μM) of Mdm2₂₅₋₁₁₇ protein to a 15 nM solution of fl-p53 in Mdm2₂₅₋₁₁₇ dialysis buffer afforded the saturation binding curve shown in FIG. 7. The IC₅₀ value obtained from this binding curve was fit into equation (1) to calculate the dissociation constant (K_{D1}) for the p53/Mdm2 complex (Roehrl et al., Biochemistry 43:16056-66 (2004)).

K_{D1}=(R_{T}*(1−F_{SB})+L_{ST}*F_{SB}²)/F_{SB}−L_{ST} (1)

where:
R_{T}=Total concentration of Mdm2 protein
L_{ST}=Total concentration of p53 fluorescent peptide
F_{SB}=Fraction of bound p53 fluorescent peptide

The K_{D1} of fl-p53 was determined to be 129±38 nM. For competition experiments, appropriate concentrations of the teHBS or HBS peptidomimetics (10 nm to 100 μM) were added to a solution of 300 nM Mdm2 and 15 nM FluP53. The resulting mixtures were incubated at 25° C. for 60 minutes before measuring the degree of dissociation of fl-p53 by polarization. The IC₅₀ was fit into equation (2) to calculate the K_{D2} value of teHBS 1 and HBS 2 (Roehrl et al., Biochemistry 43:16056-66 (2004), which is hereby incorporated by reference in its entirety).

K_{D2}=K_{D1}*F_{SB}*((L_{T}/L_{ST}*F_{SB}²−(K_{D1}+L_{ST}+R_{T})*F_{SB}+R_{T}))−1/(1−F_{SB})) (2)

where:
K_{D1}=K_{D} of fluorescent probe fl-p53
R_{T}=Total concentration of Mdm2 protein
L_{T}=Total concentration of HBS peptide
L_{ST}=Total concentration of p53 fluorescent peptide
F_{SB}=Fraction of bound p53 fluorescent peptide

### Example 14

### CD Spectroscopy

CD spectra were recorded on an AVIV 202SF CD spectrometer equipped with a temperature controller using 1 mm length cells and a scan speed of 5 nm/min. The spectra were averaged over 10 scans with the baseline subtracted from analogous conditions as that for the samples. The samples were prepared in 0.1×phosphate buffered saline (13.7 mM NaCl, 1 mM phosphate, 0.27 mM KCl, pH 7.4), containing 10% trifluoroethanol, with the final peptide concentration of 50 μM. The concentrations of unfolded peptides were determined by the UV absorption of tyrosine residue at 275 nm in 6.0 M guanidinium hydrochloride aqueous solution. The helix content of each peptide was determined from the mean residue CD at 222 nm, [θ]₂₂₂ (deg cm² dmol⁻¹) corrected for the number of amino acids. Percent helicity was calculated from the ratio [θ]₂₂₂[θ]ₘₐₓ, where [θ]ₘₐₓ=(−44000+250 T)(1−k/n)=−23,400 for k=4.0 and n=number of amino acid residues in the peptide (Luo & Baldwin, Biochemistry 36:8413-21 (1997); Shepherd et al., J. Am. Chem. Soc'y 127:2974-83 (2005); Wang et al., J. Am. Chem. Soc'y 128:9248-56 (2006)).

### Example 15

### 2D NMR Spectroscopy

All experiments were carried out on a Bruker AVANCE 500 MHz spectrometer at 25° C. Samples of teHBS 1 were prepared by dissolving 2 mg of peptide in 400 μL PBS buffer (pH 3.5) and 100 μL TFE-d₃. The pH of the solution was adjusted to 3.5 by adding 1 M HCl. The 1D proton spectra or 2D TOCSY spectra (when overlapping was severe) were employed to read the chemical shifts of the amide protons. Solvent suppression was achieved with a 3919 Watergate pulse sequence. All 2D spectra were recorded by collecting 4092 complex data points in the t2 domain by averaging 64 scans and 128 increments in the t1 domain with States-TPPI mode. All TOCSY experiments were performed with a mixing time of 80 ms on 6000 Hz spin lock frequency, and all NOESY with the mixing time of 300 ms. The data were processed and analyzed using Bruker TOPSPIN program. The original free induction decays (FIDs) were zero-filled to give a final matrix of 2048 by 2048 real data points. A 90° sine-square window function was applied in both dimensions.

### Discussion of Examples 1-15

The use of a thioether linkage (teHBS in FIG. 1) as an alternative to the all-hydrocarbon linkage of a traditional HBS was investigated. Several peptide cyclization strategies have exploited thioether formation using nucleophilic substitutions of primary alkyl halides (Roberts et al., Tetrahedron Lett. 39:8357-60 (1998); Lung et al., Lett. Peptide Sci. 6:45-49 (1999); Roberts & Ede, J. Peptide Sci. 13:811-21 (2007); Brunel & Dawson, Chem. Comm. 20:2552-54 (2005)). It was envisaged that a substitution reaction or a conjugate addition reaction would provide ready access to teHBS helices. The conditions required to affect these reactions are mild and the resulting thioether linkages have been shown to be stable in biological systems (Tugyi et al., J. Peptide Sci. 11:642-49 (2005)). Herein, the efficient synthesis of a teHBS α-helix that mimics the p53 activation domain is described. The solution conformation of the teHBS p53 helix in aqueous buffers was examined by circular dichroism and 2D NMR spectroscopies, and its potential to target Mdm2 was investigated by a fluorescence polarization competition assay. The studies described in Examples 12-15 suggest that the thioether linkage nucleates the helical conformation and targets protein receptors as well as the hydrocarbon system.

teHBS 1, an analog of a previously reported HBS helix (“HBS 2” (Patgiri et al., Acc. Chem. Res. 41:1289-300 (2008); U.S. Pat. No. 7,202,332)), was designed to compare the helicities and protein binding capabilities of the two systems (Table 5). HBS 2 mimics the p53 activation domain and has been shown to target Mdm2 with high affinity and selectivity (Henchey et al., ChemBiochem 11:2104-07 (2010)). Interaction of p53 with Mdm2 is intimately involved in regulating the crucial process of programmed cell death (Joerger & Fersht, Annu. Rev. Biochem. 77:557-82 (2008)). This complex has been targeted with several different types of synthetic inhibitors (Murray & Gellman, Biopolymers 88:657-86 (2007); Gemperli et al., J. Am. Chem. Soc'y 127:1596-97 (2005); Bernal et al., J. Am. Chem. Soc'y 129:2456-57 (2007); Lee et al., J. Am. Chem. Soc'y 133:676-79 (2011); Shangary & Wang, Clin. Cancer Res. 14:5318-24 (2008); Campbell et al., Org. Biomol. Chem. 8:2344-51 (2010); Yin et al., Angew. Chem. Int'l Ed. 44:2704-07 (2005)), making it a model protein-protein interaction for inhibitor design.

TABLE 5
Summary of Biophysical Data for HBS and teHBS p53 Helices.

| Compound | Sequence^{a} | % Helicity^{b} | K_{d} for Mdm2 (nM)^{c} |
|---|---|---|---|
| teHBS α-helix 1 | 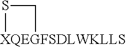 | 54 | 224 ± 20 |
| HBS α-helix 2 | 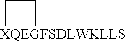 | 48 | 232 ± 34 |

^{a}X denotes pentenoic acid and thiopropionic acid residues in the HBS and teHBS macrocycles, respectively.
^{b}Values obtained from circular dichroism spectroscopy studies.
^{c}From fluorescence polarization competition assay.

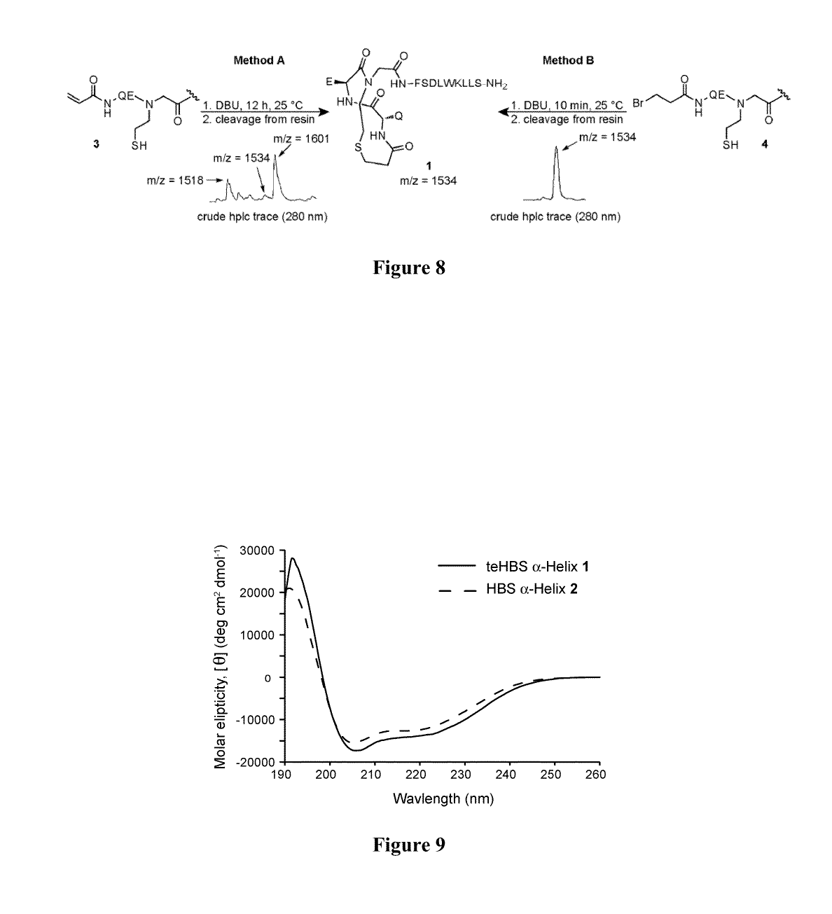

As shown in FIG. 8, two different approaches for solid-phase synthesis of teHBS α-helices were evaluated: a Michael reaction (Method A) and the nucleophilic substitution method (Method B). The precursor peptides 3 and 4 were synthesized as described in Examples 1-7. Various bases and solvents were tested with peptides 3 and 4, and model tetrapeptide sequences, to establish the optimal cyclization conditions. For example, peptide 3 or 4 was treated with 5 equivalents of triethylamine, N,N-diisopropylethylamine, n-butylamine, or DBU, in DMF, in separate reaction vessels, and each reaction was monitored periodically using a qualitative on-resin Ellman test (Ellman, Arch. Biochem. Biophys. 82:70-77 (1959); Badyal et al., Tetrahedron Lett. 42:8531-33 (2001)). After 12 hours only the DBU-catalyzed reaction indicated complete thiol consumption for 3; however, HPLC traces of the crude reaction revealed a complex mixture of products (FIG. 8). For Method B and peptide 4, DBU was again observed to be the most effective base. In this instance, HPLC and mass spectrometry analysis indicated a significant improvement in the yield of the desired product.

After identifying an efficient synthetic method, NMR and circular dichroism spectroscopies were utilized to examine the conformation of teHBS 1. Circular dichroism studies were performed in 10% trifluoroethanol in phosphate buffered saline (PBS). As expected for a canonical α-helix, double minima were observed near 208 nm and 222 nm and a maximum near 190 nm (FIG. 9). The percent helicity of teHBS 1 was estimated by the mean residue ellipticity at 222 nm to be 54%, although such assessments typically underestimate helical contents of short peptides (Wang et al., J. Am. Chem. Soc. 128:9248-56 (2006); Shepherd et al., J. Am. Chem. Soc. 127:2974-83 (2005); Chin et al., Proc. Nat'l Acad. Sci. USA 99:15416-21 (2002)). Significantly, the CD spectrum of teHBS 1 indicates that it has similar conformational stability to HBS 2 (FIG. 9). Helical content for HBS 2 was calculated to be 48%.

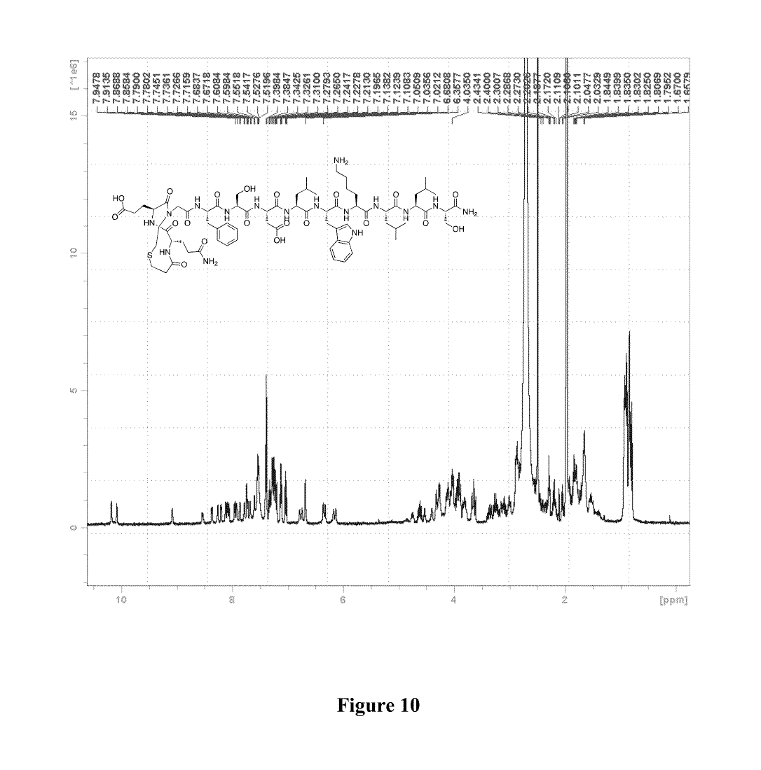

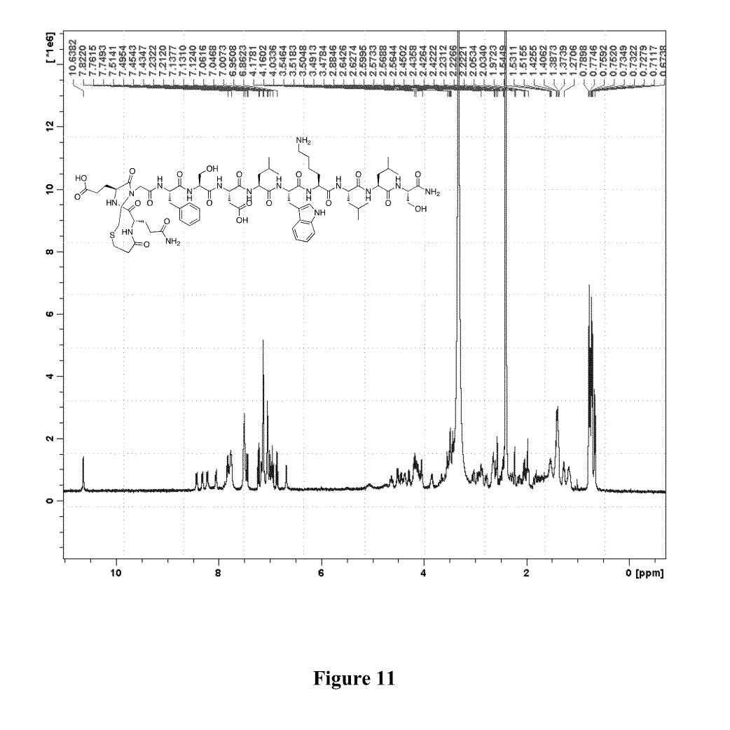

NMR spectroscopy was next utilized to obtain a detailed analysis of the peptide conformation at the atomic level. An initial 1D ¹H NMR spectrum was acquired in d₃-ACN with a 5% d₆-DMSO to enable solubility. Two sets of NMR peaks were observed in this solution, as shown in FIG. 10. When the spectrum was acquired in d₆-DMSO alone, a single set of peaks was observed, as shown in FIG. 11, indicating the presence of either two slowly equilibrating conformers in d₃-ACN/d₆-DMSO or peptide aggregation. In 20% trifluoroethanol (TFE) in 1 mM PBS (pH 3.5), two conformers were again observed with the major conformer present in a 10:1 ratio. Analysis of the NMR spectra obtained in this solution focused on the major conformer.

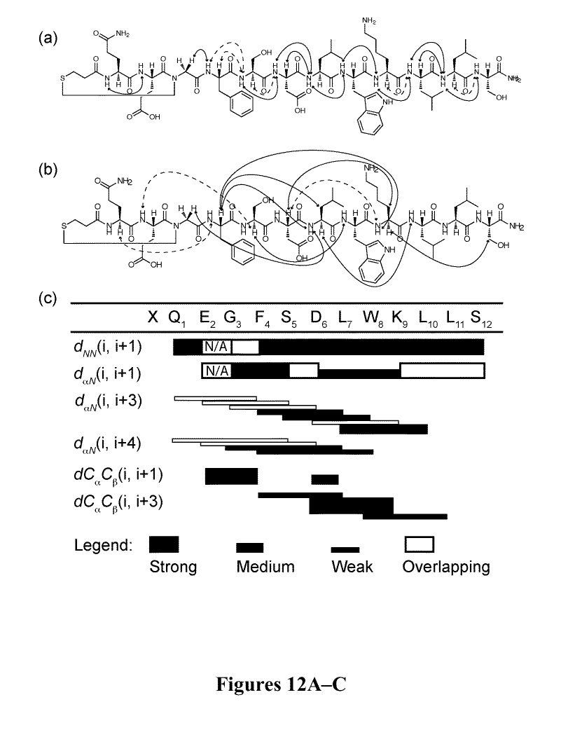

2D TOCSY and NOESY spectra of teHBS 1 enabled full assignment of the fingerprint region. Sequential NN (i and i+1) NOESY cross-peaks, a signature of helical structure, were observed for teHBS 1 as shown in the NOESY correlation chart (see FIG. 12C), although spectral overlap prevented assignment of some key cross-peaks. The NOESY spectrum further reveals several nonsequential medium range NOEs, for example, dαN(i, i+3) and dαN(i, i+4), that provide strong evidence of a helical structure (FIGS. 12A-B) (KENT WÜTHRICH, NMR OF PROTEINS AND NUCLEIC ACIDS (1986)). The ³J_{NHCHα} coupling constant provides a measure of the Φ angle and affords intimate details about the local conformation in peptides and proteins (KENT WÜTHRICH, NMR OF PROTEINS AND NUCLEIC ACIDS (1986)). The ³J_{NHCHα} values typically range between 4 and 6 Hz (−70<φ<−30) for α-helices, and a series of three or more coupling constants in this range are indicative of the α-helical structure (KENT WÜTHRICH, NMR OF PROTEINS AND NUCLEIC ACIDS (1986)). As shown in Table 6, with the exception of Q1, F4, S5, and S12, ³J_{NHCHα} coupling constants and calculated Φ angles were consistent with values expected for an α-helix. While the large values for F4 and S5 are somewhat anomalous, the value for Q1 is not unexpected because it is situated within the macrocycle. The Φ angle for S12 suggests greater flexibility near the C-terminus.

TABLE 6
³J_{NHαCH} Coupling Constants and Calculated Φ Angles.

| | Q₁ | E₂ | G₃ | F₄ | S₅^{c} | D₆^{c} | L₇ | W₈ | K₉ | L₁₀ | L₁₁ | S₁₂ |
|---|---|---|---|---|---|---|---|---|---|---|---|---|
| ³J_{NHCHα}^{a} | 9.75 | 5.15 | N/A | 6.95 | 6.45 | 4.85 | 3.70 | 3.20 | 4.35 | 4.00 | 5.15 | 7.20 |
| Φ (deg)^{b} | −120 | −68 | N/A | −81 | −78 | −65 | −55 | −56 | −61 | −58 | −68 | −84 |

^{a}J values are in Hz.
^{b}Calculated using the Karplus equation.
^{c}Coupling constants were derived from 1D ¹H NMR spectra acquired at 313 K due to overlapping resonances at 298 K.

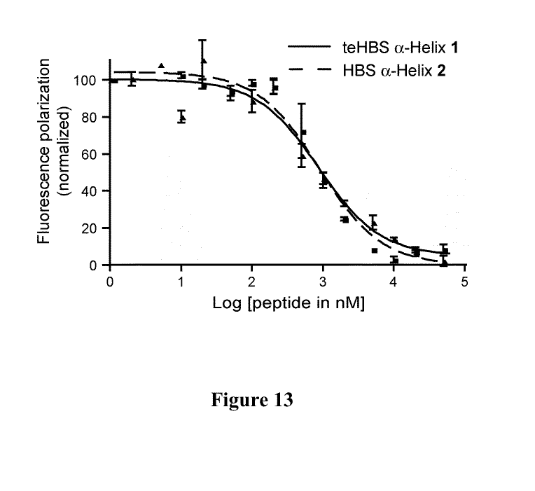

The CD and NMR data suggest that the thioether hydrogen bond surrogate can efficiently nucleate a helical conformation in the attached peptide sequence. To ascertain that the thioether linkage does not interfere with the ability of these artificial helices to target their cognate protein receptors, the abilities of teHBS 1 and HBS 2 to bind Mdm2 were compared in a fluorescence polarization competition assay (FIG. 13) (Henchey et al., ChemBiochem 11:2104-07 (2010)). Both artificial helices were found to target Mdm2 with similar affinities; the calculated K_{d} values for teHBS 1 and HBS 2 were 224±20 and 232±34 nM, respectively.

Salts, optionally pharmaceutically acceptable salts, herein unless otherwise indicated, include, for example, salts of acidic or basic groups present in compounds herein. For example, acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (e.g., 1,1′-methylene-bis-(2-hydroxy-3-naphthoate)) salts. Certain compounds herein can form pharmaceutically acceptable salts with various amino acids, including any amino acid disclosed herein. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A peptidomimetic or its salt having a stable, internally constrained protein secondary structure comprising a thioether-, ether-, or alkylamine-linked hydrogen bond surrogate.

2. The peptidomimetic or its salt according to claim 1, wherein the protein secondary structure is selected from the group consisting of an α-helix, a 3₁₀-helix, a pi helix, a gramicidin helix, a β-sheet macrocycle, and a β-hairpin.

3. The peptidomimetic or its salt according to claim 2, wherein the protein secondary structure is an α-helix.

4.-8. (canceled)

9. The peptidomimetic or its salt according to claim 1, wherein the protein secondary structure comprises an ether-linked hydrogen bond surrogate.

10. The peptidomimetic or its salt according to claim 9, wherein the ether-linked hydrogen bond surrogate is of the moiety

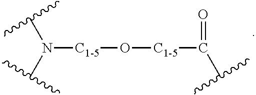 [SMILES: *C(=O)COCN(*)* ; "."]

11. The peptidomimetic or its salt according to claim 1, wherein the protein secondary structure comprises a thioether-linked hydrogen bond surrogate.

12. The peptidomimetic or its salt according to claim 11, wherein the thioether-linked hydrogen bond surrogate is of the moiety

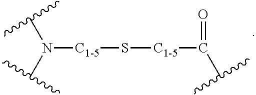 [SMILES: *C(=O)CSCN(*)* ; "."]

13. The peptidomimetic or its salt according to claim 1, wherein the protein secondary structure comprises an alkylamine-linked hydrogen bond surrogate.

14. The peptidomimetic or its salt according to claim 13, wherein the alkylamine-linked hydrogen bond surrogate is of the moiety

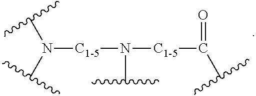 [SMILES: *C(=O)CN(*)CN(*)* ; "."]

15. The peptidomimetic or its salt according to claim 1, wherein the peptidomimetic is a compound of Formula I: 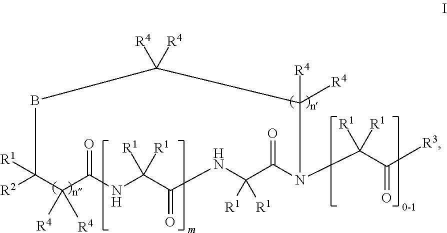
wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula 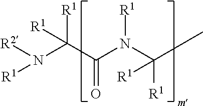
wherein:
R^{2′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
m′ is zero or any number;
R³ is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of formula 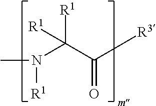
wherein:
R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; and
m″ is zero or any number;
each R⁴ is independently hydrogen, an alkyl, an alkenyl, an alkynyl, a cycloalkyl, a heterocyclyl, an aryl, a heteroaryl, or an arylalkyl; and
m, n′, and n″ are each independently zero, one, two, three, or four, wherein the sum of m, n′, and n″ is from two to six.

16. The peptidomimetic or its salt according to claim 15, wherein B is O.

17. The peptidomimetic or its salt according to claim 15, wherein B is S.

18. The peptidomimetic or its salt according to claim 15, wherein B is NR¹.

19. The peptidomimetic or its salt according to claim 15, wherein the sum of m, n′, and n″ is 2.

20. The peptidomimetic or its salt according to claim 19, wherein m is zero and the sum of n′ and n″ is 2.

21. The peptidomimetic or its salt according to claim 15, wherein the sum of m, n′, and n″ is 3.

22. The peptidomimetic or its salt according to claim 21, wherein m is 1 and the sum of n′ and n″ is 2.

23.-26. (canceled)

27. The peptidomimetic or its salt according to claim 15, wherein the peptidomimetic is a compound of Formula IA:

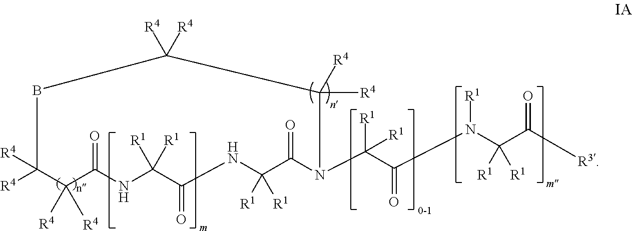

28. The peptidomimetic or its salt according to claim 15, wherein the peptidomimetic is a compound of Formula IB:

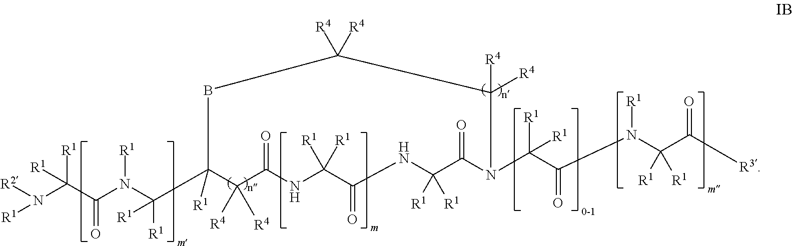

29. The peptidomimetic or its salt according to claim 15, wherein the peptidomimetic is a compound of Formula IC:

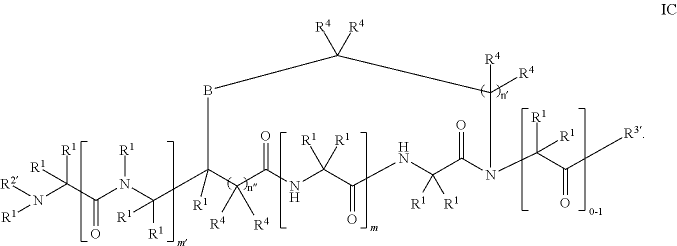

30.-37. (canceled)

38. A method of preparing a compound of Formula IA or its salt: 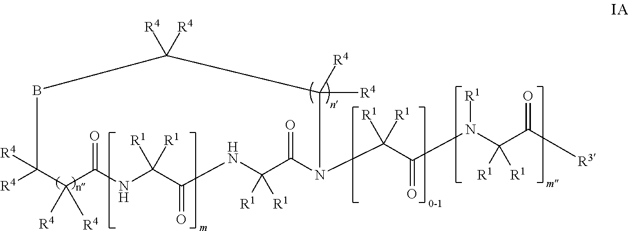
wherein:
B is O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R^{3′} is hydrogen; an alkyl; an alkenyl; an alkynyl; a cycloalkyl; a heterocyclyl; an aryl; a heteroaryl; an arylalkyl; an amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
each R⁴ is independently hydrogen, an alkyl, an alkenyl, an alkynyl, a cycloalkyl, a heterocyclyl, an aryl, a heteroaryl, or an arylalkyl;
m″ is zero or any number; and
m, n′, and n″ are each independently zero, one, two, three, or four, wherein the sum of m, n′, and n″ is from two to six;
said method comprising:
providing a compound of Formula III: 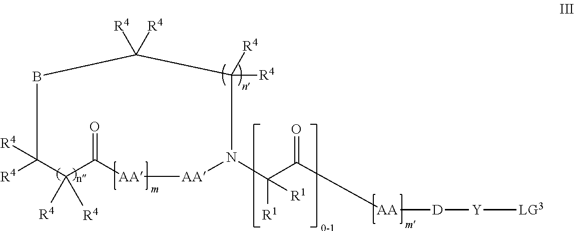
wherein:
each AA is independently a moiety of formula 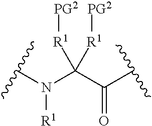
wherein each PG² is independently absent or a protecting group for protection of the R¹ to which it is attached;
each AA′ is independently a moiety of formula 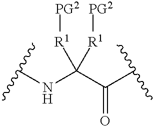
wherein each PG² is independently absent or a protecting group for protection of the R¹ to which it is attached;
D is NR¹ or 0;
LG³ is absent, a surface for solid phase synthesis, an alkyl/aryl ester, or an alkyl/aryl amide; and
Y is hydrogen, an alkyl, an alkenyl, an alkynyl, a cycloalkyl, a heterocyclyl, an aryl, a heteroaryl, an arylalkyl, or a surface for solid phase synthesis; and
reacting the compound of Formula III under conditions effective to produce a compound of Formula IA.

39-50. (canceled)

51. A composition comprising at least one of the peptidomimetic or its salt of claim 1, and further comprising an excipient or a vehicle.

52. (canceled)

53. A method of making a composition, comprising combining at least one of the peptidomimetic or its salt of claim 1 and an excipient or a vehicle.

54. A method for promoting cell death, comprising contacting a cell with one or more compounds or their salts of claim 1 that fully or partially inhibit p53/hDM2, under conditions effective for the one or more compounds or their salts to promote cell death.
